(19) United States
(12) Patent Application Publication
(10) Pub. No.: US 2010/0136105 A1
(43) Pub. Date: Jun. 3, 2010

(54) PHARMACEUTICAL COMPOSITIONS AND DOSAGE FORMS FOR ADMINISTRATION OF HYDROPHOBIC DRUGS

(75) Inventors: FENG-JING CHEN, Irvine, CA (US); MAHESH V. PATEL, SALT LAKE CITY, UT (US); DAVID T. FIKSTAD, STANFORD, CA (US); HUIPING ZHANG, MALVERN, PA (US); CHANDRASHEKAR GILIYAR, SALT LAKE CITY, UT (US)

Correspondence Address: WILSON, SONSINI, GOODRICH & ROSATI, 650 PAGE MILL ROAD, PALO ALTO, CA, 94304-1050, US

(73) Assignee: LIPOCINE, INC., Salt Lake City, UT (US)

(21) Appl. No.: 12/625,309

(22) Filed: Nov. 24, 2009

Related U.S. Application Data
(63) Continuation of application No. 10/444,935, filed on May 22, 2003.
Application No. 10/444,935 is a continuation-in-part of application No. 09/716,029, filed on Nov. 17, 2000, now Pat. No. 6,982,281.
Application No. 09/716,029 is a continuation-in-part of application No. 09/877,541, filed on Jun. 8, 2001, now Pat. No. 6,761,903.
Application No. 09/877,541 is a continuation-in-part of application No. 09/345,615, filed on Jun. 30, 1999, now Pat. No. 6,267,985.
Application No. 09/345,615 is a continuation-in-part of application No. 09/751,968, filed on Dec. 29, 2000, now Pat. No. 6,458,383.
Application No. 09/751,968 is a continuation-in-part of application No. 09/375,636, filed on Aug. 17, 1999, now Pat. No. 6,309,663.

(51) Int. Cl. A61K 38/22; A61K 9/10; A61P 5/24; A61K 9/66; A61K 9/20

(52) U.S. Cl. 424/455; 514/12; 424/489; 424/464; 977/773; 977/904

(57) ABSTRACT

The present invention relates to pharmaceutical compositions and methods for improved solubilization of triglycerides and improved delivery of therapeutic agents. Compositions of the present invention include a carrier, where the carrier is formed from a combination of a triglyceride and at least two surfactants, at least one of which is hydrophilic. Upon dilution with an aqueous medium, the carrier forms a clear, aqueous dispersion of the triglyceride and surfactants.

### CROSS-REFERENCE

This application is a continuation of Ser. No. 11/444,935, filed May 22, 2003, which is a continuation-in-part of U.S. Pat. No. 6,982,281 filed Nov. 17, 2000 and a continuation-in-part of U.S. Pat. No. 6,761,903 filed Jun. 8, 2001, which is a continuation-in-part of U.S. Pat. No. 6,267,985 filed Jun. 30, 1999, and a continuation-in-part of U.S. Pat. No. 6,458,383 filed Dec. 29, 2000, which is a continuation-in-part of U.S. Pat. No. 6,309,663 filed Aug. 17, 1999, the disclosures of which are incorporated herein by reference in their entireties.

### BACKGROUND OF TILE INVENTION

A wide variety of therapeutic agents are conventionally formulated in oil/water emulsion systems. These conventional emulsions take advantage of the increased solubility of many therapeutic agents in oils (triglycerides). Thus, one conventional approach is to solubilize a therapeutic agent in a bioacceptable triglyceride solvent, such as a digestible vegetable oil, and disperse this oil phase in an aqueous medium. The dispersion may be stabilized by emulsifying agents and provided in emulsion form. Alternatively, the therapeutic agent can be provided in a water-free formulation, with an aqueous dispersion being formed in vivo in the gastrointestinal environment. The properties of these oil-based formulations are determined by such factors as the size of the triglyceride/therapeutic agent colloidal particles and the presence or absence of surfactant additives.

### SUMMARY OF THE INVENTION

It is therefore an object of the present invention to provide pharmaceutical compositions capable of solubilizing therapeutically effective amounts of therapeutic agents. It is another object of the present invention to provide pharmaceutical compositions capable of solubilizing therapeutically effective amounts of therapeutic agents, including pharmaceutical, nutritional, and cosmeceutical agents.

It is another object of the invention to provide triglyceride-containing pharmaceutical compositions that are homogeneous and thermodynamically stable. It is still another object of the invention to provide pharmaceutical compositions of a therapeutic agent that have decreased dependence upon lipolysis for bioabsorption. It is yet another object of the invention to provide pharmaceutical compositions capable of increasing the rate and/or extent of bioabsorption of co-administered therapeutic agents.

In accordance with these and other objects and features, the present invention provides pharmaceutical compositions for improved solubilization of triglycerides, and improved delivery of therapeutic agents. It has been surprisingly found that pharmaceutical compositions containing significant amounts of triglycerides can be formed without the disadvantages of conventional triglyceride-containing compositions by using a combination of surfactants and triglycerides in amounts such that when the pharmaceutical composition is mixed with an aqueous medium, a clear aqueous dispersion is formed. Such compositions can be co-administered with a therapeutic agent to increase the rate and/or extend of bioabsorption of the therapeutic agent, or can be provided with a therapeutic agent in the preconcentrate composition or in the diluent solution.

These and other objects and features of the present invention will become more fully apparent from the following description and appended claims, or may be learned by the practice of the invention as set forth hereinafter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The present invention overcomes the problems described above characteristic of conventional triglyceride-containing formulations by providing unique pharmaceutical compositions that form clear aqueous dispersions upon mixing with an aqueous medium. Surprisingly, the present inventors have found that compositions including triglycerides and a combination of surfactants can solubilize therapeutically effective amounts of therapeutic agents. In addition, when they are mixed with an aqueous medium, the compositions are surprisingly able to form homogeneous, single-phase aqueous dispersions that are thermodynamically stable and optically clear. The optical clarity is indicative of a very small particle size within the aqueous dispersions, and this small particle size substantially reduces lipolysis dependence of the rate of bioabsorption, and other disadvantages of conventional triglyceride-containing formulations. Use of these formulations is thus believed to result in an enhanced extent, rate and/or consistency of absorption of the therapeutic agent. Advantageously, the compositions of the present invention are surprisingly able to increase solubilize greater amounts of triglycerides, than conventional formulations, even when the total surfactant concentration is the same as in a conventional formulation. In addition, the compositions of the present invention are surprisingly able to increase the solubilization power of surfactants as well.

### A. Pharmaceutical Compositions

In one embodiment, the present invention provides a pharmaceutical composition including carrier. The carrier includes a triglyceride and at least two surfactants, at least one of which is a hydrophilic surfactant. Optionally, the carrier includes a triglyceride, at least one hydrophilic surfactant, and at least one hydrophobic surfactant. The triglyceride and surfactants are present in amounts such that upon dilution with an aqueous medium, either in vitro or in vivo, the composition forms a clear aqueous dispersion. It is a particular and surprising feature of the present invention that the composition is homogeneous and optically clear, despite the presence of substantial amounts of triglycerides, thereby providing surprising and important advantages over conventional triglyceride-containing formulations.

### 1. Triglycerides

The compositions of the present invention include one or more pharmaceutically acceptable triglycerides. Examples of triglycerides suitable for use in the present invention are shown in Table 1. In general, these triglycerides are readily available from commercial sources. For several triglycerides, representative commercial products and/or commercial suppliers are listed.

TABLE 1
Triglycerides

| Triglyceride | Commercial Source |
|---|---|
| Aceituno oil | |
| Almond oil | Super Refined Almond Oil (Croda) |
| Arachis oil | |
| Babassu oil | |
| Blackcurrant seed oil | |
| Borage oil | |
| Buffalo ground oil | |
| Candlenut oil | |
| Canola oil | Lipex 108 (Abitec) |
| Castor oil | |
| Chinese vegetable tallow oil | |
| Cocoa butter | |
| Coconut oil | Pureco 76 (Abitec) |
| Coffee seed oil | |
| Corn oil | Super Refined Corn Oil (Croda) |
| Cottonseed oil | Super Refined Cottonseed Oil (Croda) |
| Crambe oil | |
| Cuphea species oil | |
| Evening primrose oil | |
| Grapeseed oil | |
| Groundnut oil | |
| Hemp seed oil | |
| Illipe butter | |
| Kapok seed oil | |
| Linseed oil | |
| Menhaden oil | Super Refined Menhaden Oil (Croda) |
| Mowrah butter | |
| Mustard seed oil | |
| Oiticica oil | |
| Olive oil | Super Refined Olive Oil (Croda) |
| Palm oil | |
| Palm kernel oil | |
| Peanut oil | Super Refined Peanut Oil (Croda) |
| Poppy seed oil | |
| Rapeseed oil | |
| Rice bran oil | |
| Safflower oil | Super Refined Safflower Oil (Croda) |
| Sal fat | |
| Sesame oil | Super Refined Sesame Oil (Croda) |
| Shark liver oil | Super Refined Shark Liver Oil (Croda) |
| Shea nut oil | |
| Soybean oil | Super Refined Soybean Oil (Croda) |
| Stillingia oil | |
| Sunflower oil | |
| Tall oil | |
| Tea seed oil | |
| Tobacco seed oil | |
| Tung oil (China wood oil) | |
| Ucuhuba Vernonia oil | |
| Wheat germ oil | Super Refined Wheat Germ Oil (Croda) |
| Hydrogenated castor oil | Castorwax |
| Hydrogenated coconut oil | Pureco 100 (Abitec) |
| Hydrogenated cottonseed oil | Dritex C (Abitec) |
| Hydrogenated palm oil | Dritex PST (Abitec); Softisan 154 (Hüls) |
| Hydrogenated soybean oil | Sterotex HM NF (Abitec); Dritex S (Abitec) |
| Hydrogenated vegetable oil | Sterotex NF (Abitec); Hydrokote M (Abitec) |
| Hydrogenated cottonseed and castor oil | Sterotex K (Abitec) |
| Partially hydrogenated soybean oil | Hydrokote AP5 (Abitec) |
| Partially soy and cottonseed oil | Apex B (Abitec) |
| Glyceryl tributyrate | (Sigma) |
| Glyceryl tricaproate | (Sigma) |
| Glyceryl tricaprylate | (Sigma) |
| Glyceryl tricaprate | Captex 1000 (Abitec) |
| Glyceryl triundecanoate | Captex 8227 (Abitec) |
| Glyceryl trilaurate | (Sigma) |
| Glyceryl trimyristate | Dynasan 114 (Hüls) |
| Glyceryl tripalmitate | Dynasan 116 (Hüls) |
| Glyceryl tristearate | Dynasan 118 (Hüls) |
| Glyceryl triarchidate | (Sigma) |
| Glyceryl trimyristoleate | (Sigma) |
| Glyceryl tripalmitoleate | (Sigma) |
| Glyceryl trioleate | (Sigma) |
| Glyceryl trilinoleate | (Sigma) |
| Glyceryl trilinolenate | (Sigma) |
| Glyceryl tricaprylate/caprate | Captex 300 (Abitec); Captex 355 (Abitec); |
| | Miglyol 810 (Hüls); Miglyol 812 (Hüls) |
| Glyceryl tricaprylate/caprate/laurate | Captex 350 (Abitec) |
| Glyceryl tricaprylate/caprate/linoleate | Captex 810 (Abitec); Miglyol 818 (Hüls) |
| Glyceryl tricaprylate/caprate/stearate | Softisan 378 (Hüls); (Larodan) |
| Glyceryl tricaprylate/laurate/stearate | (Larodan) |
| Glyceryl 1,2-caprylate-3-linoleate | (Larodan) |
| Glyceryl 1,2-caprate-3-stearate | (Larodan) |
| Glyceryl 1,2-laurate-3-myristate | (Larodan) |
| Glyceryl 1,2-myristate-3-laurate | (Larodan) |
| Glyceryl 1,3-palmitate-2-butyrate | (Larodan) |
| Glyceryl 1,3-stearate-2-caprate | (Larodan) |
| Glyceryl 1,2-linoleate-3-caprylate | (Larodan) |

Fractionated triglycerides, modified triglycerides, synthetic triglycerides, and mixtures of triglycerides are also within the scope of the invention.

Preferred triglycerides include vegetable oils, fish oils, animal fats, hydrogenated vegetable oils, partially hydrogenated vegetable oils, medium and long-chain triglycerides, and structured triglycerides. It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants,” may be suitable to provide all or part of the triglyceride component for the compositions of the present invention. Examples of commercial surfactant compositions containing triglycerides include some members of the surfactant families Gelucires (Gattefosse), Maisines (Gattefosse), and Imwitors (Huls). Specific examples of these compositions are:

Gelucire 44/14 (saturated polyglycolized glycerides);

Gelucire 50/13 (saturated polyglycolized glycerides);

Gelucire 53/10 (saturated polyglycolized glycerides);

Gelucire 33/01 (semi-synthetic triglycerides of C₈-C₁₈ saturated fatty acids);

Gelucire 39/01 (semi-synthetic glycerides); other Gelucires, such as 37/06, 43/01, 35/10, 37/02, 46/07, 48/09, 50/02, 62/05, etc.;

Maisine 35-I (linoleic glycerides); and

Imwitor 742 (caprylic/capric glycerides)

Still other commercial surfactant compositions having significant triglyceride content are known to those skilled in the art. It should be appreciated that such compositions, which contain triglycerides as well as surfactants, may be suitable to provide alt or part of the triglyceride component of the compositions of the present invention, as well as all or part of the surfactant component, as described below. Of course, none of the commonly known triglyceride-containing commercial surfactants alone provides the unique pharmaceutical compositions and characteristics as recited in the appended claims.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate. Other preferred triglycerides are saturated polyglycolized glycerides (Gelucire 44/14, Gelucire 50/13 and Gelucire 53/10), linoleic glycerides (Maisine 35-I), and caprylic/capric glycerides (Imwitor 742).

Among the preferred triglycerides, particularly preferred triglycerides include: coconut oil; corn oil; olive oil; palm oil; peanut oil; safflower oil; sesame oil; soybean oil; hydrogenated castor oil; hydrogenated coconut oil; partially hydrogenated soybean oil; glyceryl tricaprate; glyceryl trilaurate; glyceryl trioleate; glyceryl trilinoleate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides (Gelucire 44/14, Gelucire 50/13 and Gelucire 53/10); linoleic glycerides (Maisine 35-I); and caprylic/capric glycerides (Imwitor 742).

### 2. Surfactants

The carrier includes a combination of surfactants, at least one of which is a hydrophilic surfactant, with the remaining surfactant or surfactants being hydrophilic or hydrophobic. As is well known in the art, the terms “hydrophilic” and “hydrophobic” are relative terms. To function as a surfactant, a compound must necessarily include polar or charged hydrophilic moieties as well as non-polar hydrophobic (lipophilic) moieties; i.e., a surfactant compound must be amphiphilic. An empirical parameter commonly used to characterize the relative hydrophilicity and hydrophobicity of non-ionic amphiphilic compounds is the hydrophilic-lipophilic balance (the “HLB” value). Surfactants with lower HLB values are more hydrophobic, and have greater solubility in oils, whereas surfactants with higher HLB values are more hydrophilic, and have greater solubility in aqueous mediums.

Using HLB values as a rough guide, hydrophilic surfactants are generally considered to be those compounds having an HLB value greater than about 10, as well as anionic, cationic, or zwitterionic compounds for which the HLB scale is not generally applicable. Similarly, hydrophobic surfactants are compounds having an HLB value less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or hydrophobicity for use in the present invention, as described herein.

The carrier of the present invention includes at least one hydrophilic surfactant. The hydrophilic surfactant can be any surfactant suitable for use in pharmaceutical compositions. Suitable hydrophilic surfactants can be anionic, cationic, zwitterionic or non-ionic, although non-ionic hydrophilic surfactants are presently preferred. Preferably, the carrier includes a mixture of two or more hydrophilic surfactants, more preferably two or more non-ionic hydrophilic surfactants. Also preferred are mixtures of at least one hydrophilic surfactant, preferably non-ionic, and at least one hydrophobic surfactant.

The choice of specific surfactants should be made keeping in mind the particular triglycerides and optional therapeutic agents to be used in the composition, and the range of polarity appropriate for the chosen therapeutic agent. With these general principles in mind, a very broad range of surfactants is suitable for use in the present invention. Such surfactants can be grouped into the following general chemical classes detailed in the Tables herein. The HLB values given in the Tables below generally represent the HLB value as reported by the manufacturer of the corresponding commercial product. In cases where more than one commercial product is listed, the HLB value in the Tables is the value as reported for one of the commercial products, a rough average of the reported values, or a value that, in the judgment of the present inventors, is more reliable. It should be emphasized that the invention is not limited to the surfactants in the Tables, which show representative, but not exclusive, lists of available surfactants.

### 2.1. Polyethoxylated Fatty Acids

Although polyethylene glycol (PEG) itself does not function as a surfactant, a variety of PEG-fatty acid esters have useful surfactant properties. Among the PEG-fatty acid monoesters, esters of lauric acid, oleic acid, and stearic acid are especially useful. Among the surfactants of Table 2, preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Examples of polyethoxylated fatty acid monoester surfactants commercially available are shown in Table 2.

TABLE 2
PEG-Fatty Acid Monoester Surfactants

| | COMMERCIAL PRODUCT | |
|---|---|---|
| COMPOUND | (Supplier) | HLB |
| PEG 4-100 monolaurate | Crodet L series (Croda) | >9 |
| PEG 4-100 monooleate | Crodet O series (Croda) | >8 |
| PEG 4-100 monostearate | Crodet S series (Croda), Myrj | >6 |
| | Series (Atlas/ICI) | |
| PEG 400 distearate | Cithrol 4DS series (Croda) | >10 |
| PEG 100, 200, 300 monolaurate | Cithrol ML series (Croda) | >10 |
| PEG 100, 200, 300 monooleate | Cithrol MO series (Croda) | >10 |
| PEG 400 dioleate | Cithrol 4DO series (Croda) | >10 |
| PEG 400-1000 monostearate | Cithrol MS series (Croda) | >10 |
| PEG-1 stearate | Nikkol MYS-1EX (Nikko), | 2 |
| | Coster K1 (Condea) | |
| PEG-2 stearate | Nikkol MYS-2 (Nikko) | 4 |
| PEG-2 oleate | Nikkol MYO-2 (Nikko) | 4.5 |
| PEG-4 laurate | Mapeg ® 200 ML (PPG), | 9.3 |
| | Kessco ® PEG 200ML | |
| | (Stepan), LIPOPEG 2L (LIPO | |
| | Chem.) | |
| PEG-4 oleate | Mapeg ® 200 MO (PPG), | 8.3 |
| | Kessco ® PEG 200 MO | |
| | (Stepan) | |
| PEG-4 stearate | Kessco ® PEG 200 MS | 6.5 |
| | (Stephan), Hodag 20 S | |
| | (Calgene), Nikkol MYS-4 | |
| | (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem | 8.5 |
| | SpA), Kessco ® PEG 300 MO | |
| | (Stephan), Nikkol MYO-6 | |
| | (Nikko), Emulgante A6 | |
| | (Condea) | |
| PEG-7 oleate | Algon OL 70 (Auschem SpA) | 10.4 |
| PEG-6 laurate | Kessco ® PEG300 ML | 11.4 |
| | (Stepan) | |
| PEG-7 laurate | Lauridac 7 (Condea) | 13 |
| PEG-6 stearate | Kessco ® PEG300 MS | 9.7 |
| | (Stepan) | |
| PEG-8 laurate | Mapeg ® 400 ML (PPG), | 13 |
| | LIPOPEG 4DL (Lipo Chem.) | |
| PEG-8 oleate | Mapeg ® 400 MO (PPG), | 12 |
| | Emulgante A8 (Condea), | |
| | Kessco PEG 400 MO (Stepan) | |
| PEG-8 stearate | Mapeg ® 400 MS (PPG), | 12 |
| | Myrj 45 | |
| PEG-9 oleate | Emulgante A9 (Condea) | >10 |
| PEG-9 stearate | Cremophor S9 (BASF) | >10 |
| PEG-10 laurate | Nikkol MYL-10 (Nikko), | 13 |
| | Lauridac 10 (Croda) | |
| PEG-10 oleate | Nikkol MYO-10 (Nikko) | 11 |
| PEG-10 stearate | Nikkol MYS-10 (Nikko), | 11 |
| | Coster K100 (Condea) | |
| PEG-12 laurate | Kessco ® PEG 600ML | 15 |
| | (Stepan) | |
| PEG-12 oleate | Kessco ® PEG 600MO | 14 |
| | (Stepan) | |
| PEG-12 ricinoleate | (CAS #9004-97-1) | >10 |
| PEG-12 stearate | Mapeg ® 600 MS (PPG), | 14 |
| | Kessco ® PEG 600MS | |
| | (Stepan) | |
| PEG-15 stearate | Nikkol TMGS-15 (Nikko), | 14 |
| | Koster K15 (Condea) | |
| PEG-15 oleate | Nikkol TMGO-15 (Nikko) | 15 |
| PEG-20 laurate | Kessco ® PEG 1000 ML | 17 |
| | (Stepan) | |
| PEG-20 oleate | Kessco ® PEG 1000 MO | 15 |
| | (Stepan) | |
| PEG-20 stearate | Mapeg ® 1000 MS (PPG), | 16 |
| | Kessco ® PEG 1000 MS | |
| | (Stepan), Myrj 49 | |
| PEG-25 stearate | Nikkol MYS-25 (Nikko) | 15 |
| PEG-32 laurate | Kessco ® PEG 1540 ML | 16 |
| | (Stepan) | |
| PEG-32 oleate | Kessco ® PEG 1540 MO | 17 |
| | (Stepan) | |
| PEG-32 stearate | Kessco ® PEG 1540 MS | 17 |
| | (Stepan) | |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-40 stearate | Myrj 52, Emerest ® 2715 | >10 |
| | (Henkel), Nikkol MYS-40 | |
| | (Nikko) | |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet O-100 (Croda) | 18.8 |
| PEG-100 stearate | Myrj 59, Arlacel 165 (ICI) | 19 |
| PEG-200 oleate | Albunol 200 MO (Taiwan | >10 |
| | Surf.) | |
| PEG-400 oleate | LACTOMUL (Henkel), | >10 |
| | Albunol 400 MO (Taiwan | |
| | Surf.) | |
| PEG-600 oleate | Albunol 600 MO (Taiwan | >10 |
| | Surf.) | |

### 2.2 PEG-Fatty Acid Diesters

Polyethylene glycol (PEG) fatty acid diesters are also suitable for use as surfactants in the compositions of the present invention. Among the surfactants in Table 3, preferred hydrophilic surfactants include PEG-20 dilaurate, PEG-20 dioleate, PEG-20 distearate, PEG-32 dilaurate and PEG-32 dioleate. Representative PEG-fatty acid diesters are shown in Table 3.

TABLE 3
PEG-Fatty Acid Diester Surfactants

| | COMMERCIAL PRODUCT | |
|---|---|---|
| COMPOUND | (Supplier) | HLB |
| PEG-4 dilaurate | Mapeg ® 200 DL (PPG), | 7 |
| | Kessco ® PEG 200 DL | |
| | (Stepan), LIPOPEG 2-DL | |
| | (Lipo Chem.) | |
| PEG-4 dioleate | Mapeg ® 200 DO (PPG) | 6 |
| PEG-4 distearate | Kessco ® 200 DS (Stepan) | 5 |
| PEG-6 dilaurate | Kessco ® PEG 300 DL | 9.8 |
| | (Stepan) | |
| PEG-6 dioleate | Kessco ® PEG 300 DO | 7.2 |
| | (Stepan) | |
| PEG-6 distearate | Kessco ® PEG 300 DS | 6.5 |
| | (Stepan) | |
| PEG-8 dilaurate | Mapeg ® 400 DL (PPG), | 11 |
| | Kessco ® PEG 400 DL | |
| | (Stepan), LIPOPEG 4 DL | |
| | (Lipo Chem.) | |
| PEG-8 dioleate | Mapeg ® 400 DO (PPG), | 8.8 |
| | Kessco ® PEG 400 DO | |
| | (Stepan), LIPOPEG 4 DO | |
| | (Lipo Chem.) | |
| PEG-8 distearate | Mapeg ® 400 DS (PPG), CDS | 11 |
| | 400 (Nikkol) | |
| PEG-10 dipalmitate | Polyaldo 2PKFG | >10 |
| PEG-12 dilaurate | Kessco ® PEG 600 DL | 11.7 |
| | (Stepan) | |
| PEG-12 distearate | Kessco ® PEG 600 DS | 10.7 |
| | (Stepan) | |
| PEG-12 dioleate | Mapeg ® 600 DO (PPG), | 10 |
| | Kessco ® 600 DO (Stepan) | |
| PEG-20 dilaurate | Kessco ® PEG 1000 DL | 15 |
| | (Stepan) | |
| PEG-20 dioleate | Kessco ® PEG 1000 DO | 13 |
| | (Stepan) | |
| PEG-20 distearate | Kessco ® PEG 1000 DS | 12 |
| | (Stepan) | |
| PEG-32 dilaurate | Kessco ® PEG 1540 DL | 16 |
| | (Stepan) | |
| PEG-32 dioleate | Kessco ® PEG 1540 DO | 15 |
| | (Stepan) | |
| PEG-32 distearate | Kessco ® PEG 1540 DS | 15 |
| | (Stepan) | |
| PEG-400 dioleate | Cithrol 4DO series (Croda) | >10 |
| PEG-400 distearate | Cithrol 4DS series (Croda) | >10 |

### 2.3 PEG-Fatty Acid Mono- and Di-Ester Mixtures

In general, mixtures of surfactants are also useful in the present invention, including mixtures of two or more commercial surfactant products. Several PEG-fatty acid esters are marketed commercially as mixtures or mono- and diesters. Representative surfactant mixtures are shown in Table 4.

TABLE 4
PEG-Fatty Acid Mono- and Diester Mixtures

| | COMMERCIAL PRODUCT | |
|---|---|---|
| COMPOUND | (Supplier) | HLB |
| PEG 4-150 mono, dilaurate | Kessco ® PEG 200-6000 | |
| | mono, dilaurate (Stepan) | |
| PEG 4-150 mono, dioleate | Kessco ® PEG 200-6000 | |
| | mono, dioleate (Stepan) | |
| PEG 4-150 mono, distearate | Kessco ® 200-6000 mono, | |
| | distearate (Stepan) | |

### 2.4 Polyethylene Glycol Glycerol Fatty Acid Esters

Suitable PEG glycerol fatty acid esters are shown in Table 5. Among the surfactants in the Table, preferred hydrophilic surfactants are PEG-20 glyceryl laurate, PEG-30 glyceryl laurate, PEG-40 glyceryl laurate, PEG-20 glyceryl oleate, and PEG-30 glyceryl oleate.

TABLE 5
PEG Glycerol Fatty Acid Esters

| | COMMERCIAL PRODUCT | |
|---|---|---|
| COMPOUND | (Supplier) | HLB |
| PEG-20 glyceryl laurate | Tagat ® L (Goldschmidt) | 16 |
| PEG-30 glyceryl laurate | Tagat ® L2 (Goldschmidt) | 16 |
| PEG-15 glyceryl laurate | Glycerox L series (Croda) | 15 |
| PEG-40 glyceryl laurate | Glycerox L series (Croda) | 15 |
| PEG-20 glyceryl stearate | Capmul ® EMG (ABITEC), | 13 |
| | Aldo ® MS-20 KFG (Lonza) | |
| PEG-20 glyceryl oleate | Tagat ® O (Goldschmidt) | >10 |
| PEG-30 glyceryl oleate | Tagat ® O2 (Goldschmidt) | >10 |

### 2.5. Alcohol—Oil Transesterification Products

A large number of surfactants of different degrees of hydrophobicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol), and PEG-6 caprylic/capric glycerides (Softigen 767). Preferred hydrophobic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), and PEG-20 almond glycerides (Crovol A40). The latter two surfactants are reported to have HLB values of 10, which is generally considered to be the approximate borderline between hydrophilic and hydrophobic surfactants. For purposes of the present invention, these two surfactants are considered to be hydrophobic. Representative surfactants of this class suitable for use in the present invention are shown in Table 6.

TABLE 6
Transesterification Products of Oils and Alcohols

| | COMMERCIAL PRODUCT | |
|---|---|---|
| COMPOUND | (Supplier) | HLB |
| PEG-3 castor oil | Nikkol CO-3 (Nikko) | 3 |
| PEG-5, 9, and 16 castor oil | ACCONON CA series | 6-7 |
| | (ABITEC) | |
| PEG-20 castor oil | Emalex C-20 (Nihon | 11 |
| | Emulsion), Nikkol CO-20 TX | |
| | (Nikko) | |
| PEG-23 castor oil | Emulgante EL23 | >10 |
| PEG-30 castor oil | Emalex C-30 (Nihon | 11 |
| | Emulsion), Alkamuls ® EL | |
| | 620 (Rhone- Poulenc), | |
| | Incrocas 30 (Croda) | |
| PEG-35 castor oil | Cremophor EL and EL-P | |
| | (BASF), Emulphor EL, | |
| | Incrocas-35 (Croda), Emulgin | |
| | RO 35 (Henkel) | |
| PEG-38 castor oil | Emulgante EL 65 (Condea) | |
| PEG-40 castor oil | Emalex C-40 (Nihon | 13 |
| | Emulsion), Alkamuls ® EL | |
| | 719 (Rhone- Poulenc) | |
| PEG-50 castor oil | Emalex C-50 (Nihon | 14 |
| | Emulsion) | |
| PEG-56 castor oil | Eumulgin ® PRT 56 (Pulcra | >10 |
| | SA) | |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra | >10 |
| | SA) | |
| PEG-5 hydrogenated castor oil | Nikkol HCO-5 (Nikko) | 6 |
| PEG-7 hydrogenated castor oil | Simusol ® 989 (Seppic), | 6 |
| | Cremophor WO7 (BASF) | |
| PEG-10 hydrogenated castor | Nikkol HCO-10 (Nikko) | 6.5 |
| oil | | |
| PEG-20 hydrogenated castor | Nikkol HCO-20 (Nikko) | 11 |
| oil | | |
| PEG-25 hydrogenated castor | Simusol ® 1292 (Seppic), | 11 |
| oil | Cerex ELS 250 (Auschem | |
| | SpA) | |
| PEG-30 hydrogenated castor | Nikkol HCO-30 (Nikko) | 11 |
| oil | | |
| PEG-40 hydrogenated castor | Cremophor RH 40 (BASF), | 13 |
| oil | (Croda), Emulgin HRE 40 | |
| | (Henkel) Croduret | |
| PEG-45 hydrogenated castor | Cerex ELS 450 (Auschem | 14 |
| oil | Spa) | |
| PEG-50 hydrogenated castor | Emalex HC-50 (Nihon | 14 |
| oil | Emulsion) | |
| PEG-60 hydrogenated castor | Nikkol HCO-60 (Nikko), | 15 |
| oil | Cremophor RH 60 (BASF) | |
| PEG-80 hydrogenated castor | Nikkol HCO-80 (Nikko) | 15 |
| oil | | |
| PEG-100 hydrogenated castor | Nikkol HCO-100 (Nikko) | 17 |
| oil | | |
| PEG-6 corn oil | Labrafil ® M 2125 CS | 4 |
| | (Gattefosse) | |
| PEG-6 almond oil | Labrafil ® M 1966 CS | 4 |
| | (Gattefosse) | |
| PEG-6 apricot kernel oil | Labrafil ® M 1944 CS | 4 |
| | (Gattefosse) | |
| PEG-6 olive oil | Labrafil ® M 1980 CS | 4 |
| | (Gattefosse) | |
| PEG-6 peanut oil | Labrafil ® M 1969 CS | 4 |
| | (Gattefosse) | |
| PEG-6 hydrogenated palm | Labrafil ® M 2130 BS | 4 |
| kernel oil | (Gattefosse) | |
| PEG-6 palm kernel oil | Labrafil ® M 2130 CS | 4 |
| | (Gattefosse) | |
| PEG-6 triolein | Labrafil ® M 2735 CS | 4 |
| | (Gattefosse) | |
| PEG-8 corn oil | Labrafil ® WL 2609 BS | 6-7 |
| | (Gattefosse) | |
| PEG-20 corn glycerides | Crovol M40 (Croda) | 10 |
| PEG-20 almond glycerides | Crovol A40 (Croda) | 10 |
| PEG-25 trioleate | TAGAT ® TO (Goldschmidt) | 11 |
| PEG-40 palm kernel oil | Crovol PK-70 | >10 |
| PEG-60 corn glycerides | Crovol M70 (Croda) | 15 |
| PEG-60 almond glycerides | Crovol A70 (Croda) | 15 |
| PEG-4 caprylic/capric | Labrafac ® Hydro | 4-5 |
| triglyceride | (Gattefosse) | |
| PEG-8 caprylic/capric | Labrasol (Gattefosse), | >10 |
| glycerides | Labrafac CM 10 (Gattefosse) | |
| PEG-6 caprylic/capric | SOFTIGEN ® 767 (Hüls), | 19 |
| glycerides | Glycerox 767 (Croda) | |
| Lauroyl macrogol-32 | GELUCIRE 44/14 | 14 |
| glyceride | (Gattefosse) | |
| Stearoyl macrogol glyceride | GELUCIRE 50/13 | 13 |
| | (Gattefosse) | |
| Mono, di, tri, tetra esters of | SorbitoGlyceride (Gattefosse) | <10 |
| vegetable oils and sorbitol | | |
| Pentaerythrityl tetraisostearate | Crodamol PTIS (Croda) | <10 |
| Pentaerythrityl distearate | Albunol DS (Taiwan Surf.) | <10 |
| Pentaerythrityl tetraoleate | Liponate PO-4 (Lipo Chem.) | <10 |
| Pentaerythrityl tetrastearate | Liponate PS-4 (Lipo Chem.) | <10 |
| Pentaerythrityl | Liponate PE-810 (Lipo | <10 |
| tetracaprylate/tetracaprate | Chem.), Crodamol PTC | |
| | (Croda) | |
| Pentaerythrityl tetraoctanoate | Nikkol Pentarate 408 (Nikko) | |

Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants.

### 2.6. Polyglycerized Fatty Acids

Polyglycerol esters of fatty acids are also suitable surfactants for the present invention. Among the polyglyceryl fatty acid esters, preferred hydrophobic surfactants include polyglyceryl oleate (Plurol Oleique), polyglyceryl-2 dioleate (Nikkol DGDO), and polyglyceryl-10 trioleate. Preferred hydrophilic surfactants include polyglyceryl-10 laurate (Nikkol Decaglyn 1-L), polyglyceryl-10 oleate (Nikkol Decaglyn 1-O), and polyglyceryl-10 mono, dioleate (Caprol™ PEG 860). Polyglyceryl polyricinoleates (Polymuls) are also preferred hydrophilic and hydrophobic surfactants. Examples of suitable polyglyceryl esters are shown in Table 7.

TABLE 7
Polyglycerized Fatty Acids

| | COMMERCIAL PRODUCT | |
|---|---|---|
| COMPOUND | (Supplier) | HLB |
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 isostearate | Nikkol DGMIS (Nikko) | 5-7 |
| Polyglyceryl-3 oleate | Caprol ® 3GO (ABITEC), | 6.5 |
| | Drewpol 3-1-O (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), | 9 |
| | Nikkol Hexaglyn 1-O (Nikko) | |
| Polyglyceryl-10 laurate | Nikkol Decaglyn 1-L (Nikko) | 15 |
| Polyglyceryl-10 oleate | Nikkol Decaglyn 1-O (Nikko) | 14 |
| Polyglyceryl-10 stearate | Nikkol Decaglyn 1-S (Nikko) | 12 |
| Polyglyceryl-6 ricinoleate | Nikkol Hexaglyn PR-15 | >8 |
| | (Nikko) | |
| Polyglyceryl-10 linoleate | Nikkol Decaglyn 1-LN | 12 |
| | (Nikko) | |
| Polyglyceryl-6 pentaoleate | Nikkol Hexaglyn 5-O (Nikko) | <10 |
| Polyglyceryl-3 dioleate | Cremophor GO32 (BASF) | <10 |
| Polyglyceryl-3 distearate | Cremophor GS32 (BASF) | <10 |
| Polyglyceryl-4 pentaoleate | Nikkol Tetraglyn 5-O (Nikko) | <10 |
| Polyglyceryl-6 dioleate | Caprol ® 6G20 (ABITEC), | 8.5 |
| | Hodag PGO-62 (Calgene), | |
| | PLUROL OLEIQUE CC 497 | |
| | (Gattefosse) | |
| Polyglyceryl-2 dioleate | Nikkol DGDO (Nikko) | 7 |
| Polyglyceryl-10 trioleate | Nikkol Decaglyn 3-O (Nikko) | 7 |
| Polyglyceryl-10 pentaoleate | Nikkol Decaglyn 5-O (Nikko) | 3.5 |
| Polyglyceryl-10 septaoleate | Nikkol Decaglyn 7-O (Nikko) | 3 |
| Polyglyceryl-10 tetraoleate | Caprol ® 10G4O (ABITEC), | 6.2 |
| | Hodag PGO-62 (CALGENE), | |
| | Drewpol 10-4-O (Stepan) | |
| Polyglyceryl-10 | Nikkol Decaglyn 10-IS | <10 |
| decaisostearate | (Nikko) | |
| Polyglyceryl-101 decaoleate | Drewpol 10-10-O (Stepan), | 3.5 |
| | Caprol 10G10O (ABITEC), | |
| | Nikkol Decaglyn 10-O | |
| Polyglyceryl-10 mono, | Caprol ® PGE 860 (ABITEC) | 11 |
| dioleate | | |
| Polyglyceryl polyricinoleate | Polymuls (Henkel) | 3-20 |

### 2.7. Propylene Glycol Fatty Acid Esters

Esters of propylene glycol and fatty acids are suitable surfactants for use in the present invention. In this surfactant class, preferred hydrophobic surfactants include propylene glycol monolaurate (Lauroglycol FCC), propylene glycol ricinoleate (Propymuls), propylene glycol monooleate (Myverol P-06), propylene glycol dicaprylate/dicaprate (Captex® 200), and propylene glycol dioctanoate (Captex® 800). Examples of surfactants of this class are given in Table 8.

TABLE 8
Propylene Glycol Fatty Acid Esters

| | COMMERCIAL PRODUCT | |
|---|---|---|
| COMPOUND | (Supplier) | HLB |
| Propylene glycol | Capryol 90 (Gattefosse), | <10 |
| monocaprylate | Nikkol Sefsol 218 (Nikko) | |
| Propylene glycol monolaurate | Lauroglycol 90 (Gattefosse), | <10 |
| | Lauroglycol FCC (Gattefosse) | |
| Propylene glycol oleate | Lutrol OP2000 (BASF) | <10 |
| Propylene glycol myristate | Mirpyl | <10 |
| Propylene glycol monostearate | ADM PGME-03 (ADM), | 3-4 |
| | LIPO PGMS (Lipo Chem.), | |
| | Aldo ® PGHMS (Lonza) | |
| Propylene glycol hydroxy | | <10 |
| stearate | | |
| Propylene glycol ricinoleate | PROPYMULS (Henkel) | <10 |
| Propylene glycol isostearate | | <10 |
| Propylene glycol monooleate | Myverol P-O6 (Eastman) | <10 |
| Propylene glycol | Captex ® 200 (ABITEC), | >6 |
| dicaprylate/dicaprate | Miglyol ® 840 (Hüls), | |
| | Neobee ® M-20 (Stepan) | |
| Propylene glycol dioctanoate | Captex ® 800 (ABITEC) | >6 |
| Propylene glycol | LABRAFAC PG (Gattefosse) | >6 |
| caprylate/caprate | | |
| Propylene glycol dilaurate | | >6 |
| Propylene glycol distearate | Kessco ® PGDS (Stepan) | >6 |
| Propylene glycol dicaprylate | Nikkol Sefsol 228 (Nikko) | >6 |
| Propylene glycol dicaprate | Nikkol PDD (Nikko) | >6 |

### 2.8. Mixtures of Propylene Glycol Esters—Glycerol Esters

In general, mixtures of surfactants are also suitable for use in the present invention. In particular, mixtures of propylene glycol fatty acid esters and glycerol fatty acid esters are suitable and are commercially available. One preferred mixture is composed of the oleic acid esters of propylene glycol and glycerol (Arlacel 186). Examples of these surfactants are shown in Table 9.

TABLE 9
Glycerol/Propylene Glycol Fatty Acid Esters

| | COMMERCIAL PRODUCT | |
|---|---|---|
| COMPOUND | (Supplier) | HLB |
| Oleic | ATMOS 300, ARLACEL 186 | 3-4 |
| | (ICI) | |
| Stearic | ATMOS 150 | 3-4 |

### 2.9. Mono- and Diglycerides

A particularly important class of surfactants is the class of mono- and diglycerides. These surfactants are generally hydrophobic. Preferred hydrophobic surfactants in this class of compounds include glyceryl monooleate (Peceol), glyceryl 15 ricinoleate, glyceryl laurate, glyceryl dilaurate (Capmul® GDL), glyceryl dioleate (Capmul® GDO), glyceryl mono/dioleate (Capmul® GMO-K), glyceryl caprylate/caprate (Capmul® MCM), caprylic acid mono/diglycerides (Imwitor® 988), and mono- and diacetylated monoglycerides (Myvacet® 9-45). Examples of these surfactants are given in Table 10.

TABLE 10
Mono- and Diglyceride Surfactants

| | COMMERCIAL PRODUCT | |
|---|---|---|
| COMPOUND | (Supplier) | HLB |
| Monopalmitolein (C16:1) | (Larodan) | <10 |
| Monoelaidin (C18:1) | (Larodan) | <10 |
| Monocaproin (C6) | (Larodan) | <10 |
| Monocaprylin | (Larodan) | <10 |
| Monocaprin | (Larodan) | <10 |
| Monolaurin | (Larodan) | <10 |
| Glyceryl monomyristate (C14) | Nikkol MGM (Nikko) | 3-4 |
| Glyceryl monooleate (C18:1) | PECOL (Gattefosse), Hodag | 3-4 |
| | GMO-D, Nikkol MGO | |
| | (Nikko) | |
| Glyceryl monooleate | RYLO series (Danisco), | 3-4 |
| | DIMODAN series (Danisco), | |
| | EMULDAN (Danisco), | |
| | ALDO ® MO FG (Lonza), | |
| | Kessco GMO (Stepan), | |
| | MONOMULS ® series | |
| | (Henkel), TEGIN O, | |
| | DREWMULSE GMO | |
| | (Stepan), Atlas G-695 (ICI), | |
| | GMOrphic 80 (Eastman), | |
| | ADM DMG-40, 70, and 100 | |
| | (ADM), Myverol (Eastman) | |
| Glycerol monooleate/linoleate | OLICINE (Gattefosse) | 3-4 |
| Glycerol monolinoleate | Maisine (Gattefosse), | 3-4 |
| | MYVEROL 18-92, Myverol | |
| | 18-06 (Eastman) | |
| Glyceryl ricinoleate | Softigen ® 701 (Hüls), | 6 |
| | HODAG GMR-D (Calgene), | |
| | ALDO ® MR (Lonza) | |
| Glyceryl monolaurate | ALDO ® MLD (Lonza), | 6.8 |
| | Hodag GML (Calgene) | |
| Glycerol monopalmitate | Emalex GMS-P (Nihon) | 4 |
| Glycerol monostearate | Capmul ® GMS (ABITEC), | 5-9 |
| | Myvaplex (Eastman), | |
| | IMWITOR ® 191 (Hüls), | |
| | CUTINA GMS, Aldo ® MS | |
| | (Lonza), Nikkol MGS series | |
| | (Nikko) | |
| Glyceryl mono-, dioleate | Capmul ® GMO-K (ABITEC) | <10 |
| Glyceryl palmitic/stearic | CUTINA MD-A, ESTAGEL- | <10 |
| | G18 | |
| Glyceryl acetate | Lamegin ® EE (Grün au | <10 |
| | GmbH) | |
| Glyceryl laurate | Inwitor ® 312 (Hüls), | 4 |
| | Monomuls ® 90-45 (Grün au | |
| | GmbH), Aldo ® MLD | |
| | (Lonza) | |
| Glyceryl | Imwitor ® 375 (Hüls) | <10 |
| citrate/lactate/oleate/linoleate | | |
| Glyceryl caprylate | Imwitor ® 308 (Hüls), | 5-6 |
| | Capmul ® MCMC8 | |
| | (ABITEC) | |
| Glyceryl caprylate/caprate | Capmul ® MCM (ABITEC) | 5-6 |
| Caprylic acid mono, | Imwitor ® 988 (Hüls) | 5-6 |
| diglycerides | | |
| Caprylic/capric glycerides | Imwitor ® 742 (Hüls) | <10 |
| Mono- and diacetylated | Myvacet ® 9-45, Myvacet ® | 3.8-4 |
| monoglycerides | 9-40, Myvacet ® 9-08 | |
| | (Eastman), Lamegin ® | |
| | (Grunau) | |
| Glyceryl monostearate | Aldo ® MS, Arlacel 129 (ICI), | 4.4 |
| | LIPO GMS (Lipo Chem.), | |
| | Imwitor ® 191 (Hüls), | |
| | Myvaplex (Eastman) | |
| Lactic acid esters of mono, | LAMEGIN GLP (Henkel) | <10 |
| diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | <10 |
| Distearin | (Larodan) | <10 |
| Glyceryl dilaurate (C12) | Capmul ® GDL (ABITEC) | 3-4 |
| Glyceryl dioleate | Capmul ® GDO (ABITEC) | 3-4 |
| Glycerol esters of fatty acids | GELUCIRE 39/01 | 1 |
| | (Gattefosse), GELUCIRE | |
| | 43/01 (Gattefosse) | |
| | GELUCIRE 37/06 | 6 |
| | (Gattefosse) | |
| Dipalmitolein (C16:1) | (Larodan) | <10 |
| 1,2 and 1,3-diolein (C18:1) | (Larodan) | <10 |
| Dielaidin (C18:1) | (Larodan) | <10 |
| Dilinolein (C18:2) | (Larodan) | <10 |

### 2.10. Sterol and Sterol Derivatives

Sterols and derivatives of sterols are suitable surfactants for use in the present invention. These surfactants can be hydrophilic or hydrophobic. Preferred derivatives include the polyethylene glycol derivatives. A preferred hydrophobic surfactant in this class is cholesterol. A preferred hydrophilic surfactant in this class is PEG-24 cholesterol ether (Solulan C-24). Examples of surfactants of this class are shown in Table 11.

TABLE 11
Sterol and Sterol Derivative Surfactants

| | COMMERCIAL PRODUCT | |
|---|---|---|
| COMPOUND | (Supplier) | HLB |
| Cholesterol, sitosterol, | | <10 |
| lanosterol | | |
| PEG-24 cholesterol ether | Solulan C-24 (Amerchol) | >10 |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-5 (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

### 2.11. Polyethylene Glycol Sorbitan Fatty Acid Esters

A variety of PEG-sorbitan fatty acid esters are available and are suitable for use as surfactants in the present invention. In general, these surfactants are hydrophilic, although several hydrophobic surfactants of this class can be used. Among the PEG-sorbitan fatty acid esters, preferred hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80). Examples of these surfactants are shown in Table 12.

TABLE 12
PEG-Sorbitan Fatty Acid Esters

| | COMMERCIAL PRODUCT | |
|---|---|---|
| COMPOUND | (Supplier) | HLB |
| PEG-10 sorbitan laurate | Liposorb L-10 (Lipo Chem.) | >10 |
| PEG-20 sorbitan monolaurate | Tween-20 (Atlas/ICI), Crillet | 17 |
| | 1 (Croda), DACOL MLS 20 | |
| | (Condea) | |
| PEG-4 sorbitan monolaurate | Tween-21 (Atlas/ICI), Crillet | 13 |
| | 11 (Croda) | |
| PEG-80 sorbitan monolaurate | Hodag PSML-80 (Calgene), | >10 |
| | T-Maz 28 | |
| PEG-6 sorbitan monolaurate | (Nikkol GL-1 (Nikko) | 16 |
| PEG-20 sorbitan | Tween-40 (Atlas/ICI), Crillet | 16 |
| monopalmitate | 2 (Croda) | |
| PEG-20 sorbitan monostearate | Tween-60 (Atlas/ICI), Crillet | 15 |
| | 3 (Croda) | |
| PEG-4 sorbitan monostearate | Tween-61 (Atlas/ICI), Crillet | 9.6 |
| | 31 (Croda) | |
| PEG-8 sorbitan monostearate | DACOL MSS (Condea) | >10 |
| PEG-6 sorbitan monostearate | Nikkol TS106 (Nikko) | 11 |
| PEG-20 sorbitan tristearate | Tween-65 (Atlas/ICI), Crillet | 11 |
| | 35 (Croda) | |
| PEG-6 sorbitan tetrastearate | (Nikkol GS-6 (Nikko) | 3 |
| PEG-60 sorbitan tetrastearate | Nikkol GS-460 (Nikko) | 13 |
| PEG-5 sorbitan monooleate | Tween-81 (Atlas/ICI), Crillet | 10 |
| | 41 (Croda) | |
| PEG-6 sorbitan monooleate | (Nikkol TO-106 (Nikko) | 10 |
| PEG-20 sorbitan monooleate | Tween-80 (Atlas/ICI), Crillet | 15 |
| | 4 (Croda) | |
| PEG-40 sorbitan oleate | Emalex ET 8040 (Nihon | 18 |
| | Emulsion) | |
| PEG-20 sorbitan trioleate | Tween-85 (Atlas/ICI), Crillet | 11 |
| | 45 (Croda) | |
| PEG-6 sorbitan tetraoleate | (Nikkol GO-4 (Nikko) | 8.5 |
| PEG-30 sorbitan tetraoleate | (Nikkol GO-430 (Nikko) | 12 |
| PEG-40 sorbitan tetraoleate | (Nikkol GO-440 (Nikko) | 13 |
| PEG-20 sorbitan | Tween-120 (Atlas/ICI), Crillet | >10 |
| monoisostearate | 6 (Croda) | |
| PEG sorbitol hexaoleate | Atlas G-1086 (ICI) | 10 |
| PEG-6 sorbitol hexastearate | Nikkol GS-6 (Nikko) | 3 |

### 2.12. Polyethylene Glycol Alkyl Ethers

Ethers of polyethylene glycol and alkyl alcohols are suitable surfactants for use in the present invention. Preferred hydrophobic ethers include PEG-3 oleyl ether (Volpo 3) and PEG-4 lauryl ether (Brij 30). Examples of these surfactants are shown in Table 13.

TABLE 13
Polyethylene Glycol Alkyl Ethers

| | COMMERCIAL PRODUCT | |
|---|---|---|
| COMPOUND | (Supplier) | HLB |
| PEG-2 oleyl ether, oleth-2 | Brij 92/93 (Atlas/ICI) | 4.9 |
| PEG-3 oleyl ether, oleth-3 | Volpo 3 (Croda) | <10 |
| PEG-5 oleyl ether, oleth-5 | Volpo 5 (Croda) | <10 |
| PEG-10 oleyl ether, oleth-10 | Volpo 10 (Croda), Brij 96/97 | 12 |
| | (Atlas/ICI) | |
| PEG-20 oleyl ether, oleth-20 | Volpo 20 (Croda), Brij 98/99 | 15 |
| | (Atlas/ICI) | |
| PEG-4 lauryl ether, laureth-4 | Brij 30 (Atlas/ICI) | 9.7 |
| PEG-9 lauryl ether | | >10 |
| PEG-23 lauryl ether, laureth- | Brij 35 (Atlas/ICI) | 17 |
| 23 | | |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

### 2.13. Sugar Esters

Esters of sugars are suitable surfactants for use in the present invention. Preferred hydrophilic surfactants in this class include sucrose monopalmitate and sucrose monolaurate. Examples of such surfactants are shown in Table 14.

TABLE 14
Sugar Ester Surfactants

| | COMMERCIAL PRODUCT | |
|---|---|---|
| COMPOUND | (Supplier) | HLB |
| Sucrose distearate | SUCRO ESTER 7 | 3 |
| | (Gattefosse), Crodesta F-10 | |
| | (Croda) | |
| Sucrose | SUCRO ESTER 11 | 12 |
| distearate/monostearate | (Gattefosse), Crodesta F-110 | |
| | (Croda) | |
| Sucrose dipalmitate | | 7.4 |
| Sucrose monostearate | Crodesta F-160 (Croda) | 15 |
| Sucrose monopalmitate | SUCRO ESTER 15 | >10 |
| | (Gattefosse) | |
| Sucrose monolaurate | Saccharose monolaurate 1695 | 15 |
| | (Mitsubishi-Kasei) | |

### 2.14. Polyethylene Glycol Alkyl Phenols

Several hydrophilic PEG-alkyl phenol surfactants are available, and are suitable for use in the present invention. Examples of these surfactants are shown in Table 15.

TABLE 15
Polyethylene Glycol Alkyl Phenol Surfactants

| | COMMERCIAL PRODUCT | |
|---|---|---|
| COMPOUND | (Supplier) | HLB |
| PEG-10-100 nonyl phenol | Triton X series (Rohm & | >10 |
| | Haas), Igepal CA series (GAF, | |
| | USA), Antarox CA series | |
| | (GAF, UK) | |
| PEG-15-100 octyl phenol | Triton N-series (Rohm & | >10 |
| ether | Haas), Igepal CO series (GAF, | |
| | USA), Antarox CO series | |
| | (GAF, UK) | |

### 2.15. Polyoxyethylene-Polyoxypropylene Block Copolymers

The POE-POP block copolymers are a unique class of polymeric surfactants. The unique structure of the surfactants, with hydrophilic POE and hydrophobic POP moieties in well-defined ratios and positions, provides a wide variety of surfactants suitable for use in the present invention. These surfactants are available under various trade names, including Synperonic PE series (ICI; Pluronic™ series (BASF), Emkalyx, Lutrol (BASF), Supronic, Monolan, Pluracare, and Plurodac. The generic term for these polymers is “poloxamer” (CAS 9003-11-6). These polymers have the formula:

HO(C₂H₄O)ₐ(C₃H₆O)_{b}(C₂H₄O)ₐH

where “a” and “b” denote the number of polyoxyethylene and polyoxypropylene units, respectively.

Preferred hydrophilic surfactants of this class include Poloxamers 108, 188, 217, 238, 288, 338, and 407. Preferred hydrophobic surfactants in this class include Poloxamers 124, 182, 183, 212, 331, and 335.

Examples of suitable surfactants of this class are shown in Table 16. Since the compounds are widely available, commercial sources are not listed in the Table. The compounds are listed by generic name, with the corresponding “a” and “b” values.

TABLE 16
POE-POP Block Copolymers

| | a, b values in | |
|---|---|---|
| COMPOUND | HO(C₂H₄O)ₐ(C₃H₆O)_{b}(C₂H₄O)ₐH | HLB |
| Poloxamer 105 | a = 11 b = 16 | 8 |
| Poloxamer 108 | a = 46 b = 16 | >10 |
| Poloxamer 122 | a = 5 b = 21 | 3 |
| Poloxamer 123 | a = 7 b = 21 | 7 |
| Poloxamer 124 | a = 11 b = 21 | >7 |
| Poloxamer 181 | a = 3 b = 30 | |
| Poloxamer 182 | a = 8 b = 30 | 2 |
| Poloxamer 183 | a = 10 b = 30 | |
| Poloxamer 184 | a = 13 b = 30 | |
| Poloxamer 185 | a = 19 b = 30 | |
| Poloxamer 188 | a = 75 b = 30 | 29 |
| Poloxamer 212 | a = 8 b = 35 | |
| Poloxamer 215 | a = 24 b = 35 | |
| Poloxamer 217 | a = 52 b = 35 | |
| Poloxamer 231 | a = 16 b = 39 | |
| Poloxamer 234 | a = 22 b = 39 | |
| Poloxamer 235 | a = 27 b = 39 | |
| Poloxamer 237 | a = 62 b = 39 | 24 |
| Poloxamer 238 | a = 97 b = 39 | |
| Poloxamer 282 | a = 10 b = 47 | |
| Poloxamer 284 | a = 21 b = 47 | |
| Poloxamer 288 | a = 122 b = 47 | >10 |
| Poloxamer 331 | a = 7 b = 54 | 0.5 |
| Poloxamer 333 | a = 20 b = 54 | |
| Poloxamer 334 | a = 31 b = 54 | |
| Poloxamer 335 | a = 38 b = 54 | |
| Poloxamer 338 | a = 128 b = 54 | |
| Poloxamer 401 | a = 6 b = 67 | |
| Poloxamer 402 | a = 13 b = 67 | |
| Poloxamer 403 | a = 21 b = 67 | |
| Poloxamer 407 | a = 98 b = 67 | |

### 2.16. Sorbitan Fatty Acid Esters

Sorbitan esters of fatty acids are suitable surfactants for use in the present invention. Among these esters, preferred hydrophobic surfactants include sorbitan monolaurate (Arlacel 20), sorbitan monopalmitate (Span-40), sorbitan monooleate (Span-80), sorbitan monostearate, and sorbitan tristearate. Examples of these surfactants are shown in Table 17.

TABLE 17
Sorbitan Fatty Acid Ester Surfactants

| | COMMERCIAL PRODUCT | |
|---|---|---|
| COMPOUND | (Supplier) | HLB |
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 | |
| | (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 | |
| | (Nikko) | |
| Sorbitan trioleate | Span-85 (Atlas/ICI), Crill 45 | 4.3 |
| | (Croda), Nikkol SO-30 | |
| | (Nikko) | |
| Sorbitan sesquioleate | Arlacel-C (ICI), Crill 43 | 3.7 |
| | (Croda), Nikkol SO-15 | |
| | (Nikko) | |
| Sorbitan tristearate | Span-65 (Atlas/ICI) Crill 35 | 2.1 |
| | (Croda), Nikkol SS-30 | |
| | (Nikko) | |
| Sorbitan monoisostearate | Crill 6 (Croda), Nikkol SI-10 | 4.7 |
| | (Nikko) | |
| Sorbitan sesquistearate | Nikkol SS-15 (Nikko) | 4.2 |

### 2.17. Lower Alcohol Fatty Acid Esters

Esters of lower alcohols (C₂ to C₄) and fatty acids (C₈ to C₁₈) are suitable surfactants for use in the present invention. Among these esters, preferred hydrophobic surfactants include ethyl oleate (Crodamol EO), isopropyl myristate (Crodamol IPM), and isopropyl palmitate (Crodamol IPP). Examples of these surfactants are shown in Table 18.

TABLE 18
Lower Alcohol Fatty Acid Ester Surfactants

| | COMMERCIAL PRODUCT | |
|---|---|---|
| COMPOUND | (Supplier) | HLB |
| Ethyl oleate | Crodamol EO (Croda), Nikkol | <10 |
| | EOO (Nikko) | |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

### 2.18. Ionic Surfactants

Ionic surfactants, including cationic, anionic and zwitterionic surfactants, are suitable hydrophilic surfactants for use in the present invention. Preferred anionic surfactants include fatty acid salts and bile salts. Preferred cationic surfactants include carnitines. Specifically, preferred ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Examples of such surfactants are shown in Table 19. For simplicity, typical counterions are shown in the entries in the Table. It will be appreciated by one skilled in the art, however, that any bioacceptable counterion may be used. For example, although the fatty acids are shown as sodium salts, other cation counterions can also be used, such as alkali metal cations or ammonium. Unlike typical non-ionic surfactants, these ionic surfactants are generally available as pure compounds, rather than commercial (proprietary) mixtures. Because these compounds are readily available from a variety of commercial suppliers, such as Aldrich, Sigma, and the like, commercial sources are not generally listed in the Table.

TABLE 19
Ionic Surfactants

| COMPOUND | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium | |
| docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glyco cheno deoxycholate | |
| Sodium cholylsarcosinate | |
| Sodium N-methyl taurocholate | |
| Sodium lithocholate | |
| PHOSPHOLIPIDS | |
| Egg/Soy lecithin [Epikuron ™ (Lucas Meyer), | |
| Ovothin ™ (Lucas Meyer)] | |
| Lyso egg/soy lecithin | |
| Hydroxylated lecithin | |
| Lysophosphatidylcholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl | |
| ether phosphate | |
| Esterification products of fatty alcohols or fatty | |
| alcohol ethoxylates with phosphoric acid or | |
| anhydride | |
| CARBOXYLATES | |
| Ether carboxylates (by oxidation of terminal | |
| OH group of fatty alcohol ethoxylates) | |
| Succinylated monoglycerides [LAMEGIN ZE | |
| (Henkel)] | |
| Sodium stearyl fumarate | |
| Stearoyl propylene glycol hydrogen succinate | |
| Mono/diacetylated tartaric acid esters of mono- | |
| and diglycerides | |
| Citric acid esters of mono-, diglycerides | |
| Glyceryl-lacto esters of fatty acids (CFR ref. | |
| 172.852) | |
| ACYL LACTYLATES | |
| lactylic esters of fatty acids | |
| calcium/sodium stearoyl-2-lactylate | |
| calcium/sodium stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamideo-MEA- | |
| sulfosuccinate | |
| CATIONIC SURFACTANTS | >10 |
| Lauroyl carnitine | |
| Palmitoyl carnitine | |
| Myristoyl carnitine | |
| Hexadecyl triammonium bromide | |
| Decyl trimethyl ammonium bromide | |
| Cetyl trimethyl ammonium bromide | |
| Dodecyl ammonium chloride | |
| Alkyl benzyldimethylammonium salts | |
| Diisobutyl phenoxyethoxydimethyl | |
| benzylammonium salts | |
| Alkylpyridinium salts | |
| BETAINES (TRIALKYLGLYCINE) | |
| Lauryl betaine (N-lauryl,N,N-dimethylglycine) | |
| ETHOXYLATED AMINES | |
| Polyoxyethylene-15 coconut amine | |

### 2.19. Unionized Ionizable Surfactants

Ionizable surfactants, when present in their unionized (neutral, non-salt) form, are hydrophobic surfactants suitable for use in the compositions and methods of the present invention. Particular examples of such surfactants include free fatty acids, particularly C₆-C₂₂ fatty acids, and bile acids. More specifically, suitable unionized ionizable surfactants include the free fatty acid and bile acid forms of any of the fatty acid salts and bile salts shown in Table 19.

### 2.20 Preferred Surfactants and Surfactant Combinations

Among the above-listed surfactants, several combinations are preferred. In all of the preferred combinations, the carrier includes at least one hydrophilic surfactant. Preferred non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters, sugar ethers; sucroglycerides; and mixtures thereof.

More preferably, the non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglyceryl fatty acid esters; polyoxyethylene glycerides; polyoxyethylene vegetable oils; and polyoxyethylene hydrogenated vegetable oils. The glyceride can be a monoglyceride, diglyceride, triglyceride, or a mixture.

Also preferred are non-ionic hydrophilic surfactants that are reaction mixtures of polyols and fatty acids, glycerides, vegetable oils, hydrogenated vegetable oils or sterols. These reaction mixtures are largely composed of the transesterification products of the reaction, along with often complex mixtures of other reaction products. The polyol is preferably glycerol, ethylene glycol, polyethylene glycol, sorbitol, propylene glycol, pentaerythritol, or a saccharide.

Several particularly preferred carrier compositions are those which include as a non-ionic hydrophilic surfactant PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, or a poloxamer.

Among these preferred surfactants, more preferred are PEG-20 laurate, PEG-20 oleate, PEG-35 castor oil, PEG-40 palm kernel oil, PEG-40 hydrogenated castor oil, PEG-60 corn oil, PEG-25 glyceryl trioleate, polyglyceryl-10 laurate, PEG-6 caprate/caprylate glycerides, PEG-8 caprate/caprylate glycerides, PEG-30 cholesterol, polysorbate 20, polysorbate 80, POE-9 lauryl ether, POE-23 lauryl ether, POE-10 oleyl ether, PEG-24 cholesterol, sucrose monostearate, sucrose monolaurate and poloxamers. Most preferred are PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, PEG-24 cholesterol, and hydrophilic poloxamers.

The hydrophilic surfactant can also be, or include as a component, an ionic surfactant. Preferred ionic surfactants include alkyl ammonium salts; bile acids and salts, analogues, and derivatives thereof; fusidic acid and derivatives thereof; fatty acid conjugates of amino acids, oligopeptides, and polypeptides; glyceride esters of amino acids, oligopeptides, and polypeptides; acyl lactylates; mono- and diacetylated tartaric acid esters of mono- and diglycerides; succinylated monoglycerides; citric acid esters of mono- and diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

More preferable ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono- and diacetylated tartaric acid esters of mono- and diglycerides; succinylated monoglycerides; citric acid esters of mono- and diglycerides; carnitines; and mixtures thereof.

More specifically, preferred ionic surfactants are lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Particularly preferred ionic surfactants are lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

The carrier of the present compositions includes at least two surfactants, at least one of which is hydrophilic. In one embodiment, the present invention includes at two surfactants that are hydrophilic, and preferred hydrophilic surfactants are listed above. In another embodiment, the carrier includes at least one hydrophilic surfactant and at least one hydrophobic surfactant. In this embodiment, preferred hydrophobic surfactants are alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; and polyoxyethylene hydrogenated vegetable oils.

As with the hydrophilic surfactants, hydrophobic surfactants can be reaction mixtures of polyols and fatty acids, glycerides, vegetable oils, hydrogenated vegetable oils, and sterols.

Preferably, the hydrophobic surfactant is selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid esters of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols.

More preferred are lower alcohol fatty acids esters; polypropylene glycol fatty acid esters; propylene glycol fatty acid esters; glycerol fatty acid esters; acetylated glycerol fatty acid esters; lactic acid esters of mono/diglycerides; sorbitan fatty acid esters; polyoxyethylene vegetable oils; and mixtures thereof, with glycerol fatty acid esters and acetylated glycerol fatty acid esters being most preferred. Among the glycerol fatty acid esters, the esters are preferably mono- or diglycerides, or mixtures of mono- and diglycerides, where the fatty acid moiety is a C₆ to C₂₂ fatty acid.

Also preferred are hydrophobic surfactants that are the reaction mixture of polyols and fatty acids, glycerides, vegetable oils, hydrogenated vegetable oils, and sterols. Preferred polyols are polyethylene glycol, sorbitol, propylene glycol, and pentaerythritol.

Specifically preferred hydrophobic surfactants include myristic acid; oleic acid; lauric acid; stearic acid; palmitic acid; PEG 1-4 stearate; PEG 2-4 oleate; PEG-4 dilaurate; PEG-4 dioleate; PEG-4 distearate; PEG-6 dioleate; PEG-6 distearate; PEG-8 dioleate; PEG 3-16 castor oil; PEG 5-10 hydrogenated castor oil; PEG 6-20 corn oil; PEG 6-20 almond oil; PEG-6 olive oil; PEG-6 peanut oil; PEG-6 palm kernel oil; PEG-6 hydrogenated palm kernel oil; PEG-4 capric/caprylic triglyceride, mono, di, tri, tetra esters of vegetable oil and sorbitol; pentaerythrityl di, tetra stearate, isostearate, oleate, caprylate, or caprate; polyglyceryl 2-4 oleate, stearate, or isostearate; polyglyceryl 4-10 pentaoleate; polyglyceryl-3 dioleate; polyglyceryl-6 dioleate; polyglyceryl-10 trioleate; polyglyceryl-3 distearate; propylene glycol mono- or diesters of a C₆ to C₂₀ fatty acid; monoglycerides of C₆ to C₂₀ fatty acids; acetylated monoglycerides of C₆ to C₂₀ fatty acids; diglycerides C₆ to C₂₀ fatty acids; lactic acid derivatives of monoglycerides; lactic acid derivatives of diglycerides; cholesterol; phytosterol; PEG 5-20 soya sterol; PEG-6 sorbitan tetra, hexastearate; PEG-6 sorbitan tetraoleate; sorbitan monolaurate; sorbitan monopalmitate; sorbitan mono, trioleate; sorbitan mono, tristearate; sorbitan monoisostearate; sorbitan sesquioleate; sorbitan sesquistearate; PEG 2-5 oleyl ether; POE 2-4 lauryl ether; PEG-2 cetyl ether; PEG-2 stearyl ether; sucrose distearate; sucrose dipalmitate; ethyl oleate; isopropyl myristate; isopropyl palmitate; ethyl linoleate; isopropyl linoleate; and poloxamers.

Among the specifically preferred hydrophobic surfactants, most preferred are oleic acid; lauric acid; glyceryl monocaprate; glyceryl monocaprylate; glyceryl monolaurate; glyceryl monooleate; glyceryl dicaprate; glyceryl dicaprylate; glyceryl dilaurate; glyceryl dioleate; acetylated monoglycerides; propylene glycol oleate; propylene glycol laurate; polyglyceryl-3 oleate; polyglyceryl-6 dioleate; PEG-6 corn oil; PEG-20 corn oil; PEG-20 almond oil; sorbitan monooleate; sorbitan monolaurate; POE-4 lauryl ether; POE-3 oleyl ether; ethyl oleate; and poloxamers.

### 3. Therapeutic Agent

In the embodiments of the present invention that include therapeutic agents, the therapeutic agents suitable for use in the pharmaceutical compositions and methods of the present invention are not particularly limited, as the compositions are surprisingly capable of solubilizing and delivering a wide variety of therapeutic agents. The therapeutic agents can be hydrophilic, lipophilic, amphiphilic or hydrophobic, and can be solubilized in the carrier, e.g., the triglyceride, the surfactant(s), or both the triglyceride and the surfactant, or present in the diluent. Optionally, the therapeutic agent can be present in a first, solubilized amount, and a second, non-solubilized (suspended) amount. Such therapeutic agents can be any agents having therapeutic or other value when administered to an animal, particularly to a mammal, such as drugs, nutrients, and cosmetics (cosmeceuticals). It should be understood that while the invention is described with particular reference to its value in the form of aqueous dispersions, the invention is not so limited. Thus, drugs, nutrients or cosmetics which derive their therapeutic or other value from, for example, topical or transdermal administration, are still considered to be suitable for use in the present invention.

Specific non-limiting examples of therapeutic agents that can be used in the pharmaceutical compositions of the present invention include analgesics and anti-inflammatory agents, anti-helmintics, anti-arrhythmic agents, anti-asthma agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents and immunosuppressants, anti-protozoal agents, anti-thyroid agents, anti-tussives, anxiolytic, sedatives, hypnotics and neuroleptics, β-Blockers, cardiac inotropic agents, corticosteroids, diuretics, anti-parkinsonian agents, gastro-intestinal agents, histamine H₁-receptor antagonists, keratolytics, lipid regulating agents, muscle relaxants, polysaccharide drugs, anti-anginal agents, nutritional agents, analgesics, sex hormones, stimulants, peptides, peptidomimetics, DNA, RNA, oligodeoxynucleotides, genetic material, proteins, oligonucleotides, and vaccines.

In one embodiment, the therapeutic agent is a nutritional agent. In another embodiment, the therapeutic agent is a cosmeceutical agent. In another embodiment, the therapeutic agent is a protein, peptide or oligonucleotide. In a particular aspect of this embodiment, the therapeutic agent is a protein, peptidomimetic, DNA, RNA, oligodeoxynucleotide, genetic material, peptide or oligonucleotide having a molecular weight of less than about 1000 g/mol.

In another embodiment, the therapeutic agent is hydrophobic. Hydrophobic therapeutic agents are compounds with little or no water solubility. Intrinsic water solubilities (i.e., water solubility of the unionized form) for hydrophobic therapeutic agents are less than about 1% by weight, and typically less than about 0.1% or 0.01% by weight. In a particular aspect of this embodiment, the therapeutic agent is a hydrophobic drug. In another particular aspect, the therapeutic agent is a hydrophobic drug having a molecular weight of less than about 1000 g/mol.

In another embodiment, the therapeutic agent is hydrophilic. Amphiphilic therapeutic agents are included within the class of hydrophilic therapeutic agents. Apparent water solubilities for hydrophilic therapeutic agents generally have an aqueous solubility greater than about 100 μg/ml. Such drugs include polysaccharides and other macromolecular drugs such as peptides, proteins, peptidomimetics, cytokines, nucleotides, nucleosides, genetic materials, toxoids, serum vaccines, etc. Generally, the hydrophilic drug is a polysaccharide drug, e.g., a disaccharide, oligosaccharide, or longer chain saccharide polymer that is suitable for administration to a human being. Examples of polysaccharide drugs include, without limitation, glucosamine, glycosaminoglycans, dextran, xylan, pentasaccharide, polygalacturonic acid, polymannuronic acid, chitin, pharmaceutically acceptable salts, esters or other derivatives thereof, and combinations of any of the foregoing. That is, a single polysaccharide drug may be administered, or two or more polysaccharide drugs may be administered in combination. The polysaccharide drugs may also be fragments of naturally occurring or synthetic polysaccharides.

In another embodiment, the therapeutic agent is a steroid. Steroids are compounds based on the cyclopenta[α]phenanthrene structure. Examples of steroids which have been shown to be suitable for the current invention include those with the androstane structure. Examples of such androstane steroids include cetadiol, clostebol, danazol, dehydroepiandrosterone (DHEA) (also, prasterone or dehydroisoandrosterone), DHEA sulfate, dianabol, dutasteride, exemestane, finasteride, nerobol, oxymethol one, stanolone, stanozolol, testosterone, 17-alpha-methyltestosterone, and methyltestosterone enanthate.

Another group steroids, which have been shown to be suitable, are those based on the cholane or cholesterol structure. Examples of such steroids are brassicasterol, campesterol, chenodeoxycholic acid, clionasterol, desmosterol, lanosterol, poriferasterol, α-sitosterol-, stigmasterol, and ursodeoxycholic acid.

Another suitable class of steroids for use in the present invention are those steroids based on the estrane structure. Examples of such estranes include desogestrel, equilin, 17-alpha-dihydroequilin, 17-beta-dihydroequilin, 17-alpha-estradiol, 17-beta-estradiol (estradiol), ethinyl estradiol, estriol, estrone, levonorgestrel, lynestrenol, mestranol, mibolerone, mifegyne, mifepristone, nandrolone, norethindrone (or norethistrone), norethindrone acetate (or norethisterone acetate), nortestosterone.

Also suitable is the steroid class based on the pregnane structure. Examples of such pregnanes include alfaxalone, beclomethasone, budesonide, clobetasol, clobetasone, corticosterone, desoxycorticosterone, cortisol, cortisone, dihydrocortisone, cyproterone, desonide, dexamethasone, eplerenone, epoxypregnenolone, flumethasone, megestrol, melengestrol, prednisolone, prednisone, pregnanediol, pregnanolone, pregnenolone, allopregnanolone, epiallopregnanolone, progesterone, medroxyprogesterone, spironolactone, and tibolone.

It is to be understood that steroids suitable for the present invention are not limited to those disclosed herein and include any secondary steroids, such as for example, vitamin D.

Steroid esters, such as the acetate, benzoate, cypionate, decanoate, enanthate, hemisuccinate, hexahydrobenzoate, 4-methylvalerate, propionate, stearate, valerate, and undecanoate esters would also be suitable for the present invention.

Although the invention is not limited thereby, examples of therapeutic agents not previously listed that are suitable for use in the compositions and methods of the present invention include the following representative compounds, as well as their pharmaceutically acceptable salts, isomers, esters, ethers and other derivatives: abacavir, acarbose, acebutolol, acetazolamide, acetohexamide, acrivastine, acutretin, acyclovir, alatrofloxacin, albendazole, albuterol, aldlofenac, alendronate, allopurinol, aloxiprin, alprazolam, alprenolol, alprostadil, amantadine, amiloride, aminoglutethimide, amiodarone, amiodarone HCl, amitriptyline, amlodipine, amodiaquine, amoxapine, amoxapine, amphetamine, amphotericin, amprenavir, amrinone, amsacrine, amyl nitrate, amylobarbital, amylobarbitone, aspirin, astemizole, atenolol, atorvastatin, atovaquone, atropine, auranofin, azapropazone, azathioprine, azelastine, azithromycin, baclofen, barbital, barbitone, becaplermin, beclamide, beclomethasone, bendrofluazide, benethamine, benethamine penicillin, benezepril, benidipine, benorylate, bentazepam, benzhexol, benzhexol HCl, benznidazole, benzonatate, benztropine, bephenium hydroxynaphthoate, betamethasone, bezafibrate, bicalutamide, biperiden, bisacodyl, bisanthrene, bovine growth hormone, bromazepam, bromfenac, bromocriptine, bromocriptine mesylate, bromperidol, brompheniramine, brotizolam, budesonide, bumetanide, bupropion, busulphan, butenafine, butenafine HCl, butobarbital, butobarbitone, butoconazole, butoconazole nitrate, calcifediol, calciprotiene, calcitonin, calcitriol, cambendazole, camptothecan, camptothecin, candesartan, capecitabine, capsacin, capsaicin, captopril, carbamazepine, carbimazole, carbinoxamine, carbromal, carotenes, cefazolin, cefoxitin sodium, celecoxib, cephadrine, cephalexin, cerivistatin, cetrizine, chlopheniramine, chlophenisamine, chloproguanil, chlorambucil, chlordiazepoxide, chlormethiazole, chloroquine, chlorothiazide, chlorproguanil HCl, chlorpromazine, chlorpropamide, chlorprothiocene, chlorprothixene, chlorthalidone, cholecalciferol, cilostazol, cimetidine, cinnarizine, cinoxacin, ciprofloxacin, ciprofloxacin HCl, cisapride, citalopram, citrizine, clarithromycin, clemastine, clemastine fumarate, clemizole, clenbuterol, clinofibrate, clioquinol, clobazam, clofazimine, clofibrate, clomiphene, clomiphene citrate, clomipramine, clonazepam, clopidrogel, clotiazepam, clotrimazole, cloxacillin, clozapine, codeine, conjugated estrogens, cortisone acetate, cortisone acetate, cromolyn sodium, cromoglicate, cromolyn, cyclizine, cyclosporin, cyproheptadine, cyproheptadine HCl, dacarbazine, danazol, dantrolene, dantrolene sodium, darodipine, decoquinate, delavirdine, demeclocycline, desoxymethasone, dexamphetamine, dexchlopheniramine, dexfenfluramine, dextropropyoxyphene, diamorphine, diazepam, diazoxide, dichlorophen, diclofenac, dicloxacillin, dicoumarol, dicumarol, didanosine, diethylpropion, diflunisal, digitoxin, digoxin, dihydro epiandrosterone, dihydrocodeine, dihydroergotamine, dihydroergotamine mesylate, dihydrotachysterol, diiodohydroxyquinoline, dilitazem, dilitazem HCl, diloxanide furoate, dimenhydrinate, dinitolmide, diphenhydramine, diphenooxylate, diphenoxylate HCl, diphenyl imidazole, diphenylpyrallin, dipyridamole, dirithromycin, disopyramide, divalproen, docusate, dolasetron, domperidone, donepezil, donepezil HCl, doxazosin, doxazosin doxycycline, dronabinol, droperidol, econazole, econazole nitrate, editronate, efavirenz, elanapril, ellipticine, enalapril, enkephalin, enoxacin, enoximone, enrofloxacin, epalrestate, eperisone, ephedrine, eposartan, eposartan losartan, ergocalciferol, ergotamine, ergotamine tartrate, erythromycin, erythropoietin, essential fatty acids, estramustine, ethacrynic acid, ethambutol, ethinamate, ethinyloestradiol, ethionamide, ethopropazine, ethopropazine HCl, ethotoin, etodolac, etoperidone, etoposide, etretinate, famcyclovir, famotidine, felbamate, felodipine, fenbendazole, fenbufen, fenfluramine, fenofibrate, fenolclopam, fenoldopam, fenoprofen, fenoprofen calcium, fentanyl, fexofenadine, finasteride, flecainide, flecainide acetate, fluconazole, flucortolone, flucytosine, fludrocortisone, fludrocortisone acetate, fluexetine HCl, flunanisone, flunarizine, flunarizine HCl, flunisolide, flunitrazepam, fluopromazine, fluoxetine, fluoxymisterone, flupenthixol decanoate, flupentixol, flupentixol decanoate, fluphenazine, fluphenazine decanoate, flurazepam, flurbiprofen, flurithromycin, fluticasone propionate, fluvastatin, foscarnet sodium, fosinopril, fosphenytoin, fosphenytoin sodium, frovatriptan, frusemide, fumagillin, furazolidone, furosemide, furzolidone, gabapentin, gancyclovir, gemfibrozil, gentamycin, glibenclamide, gliclazide, glipizide, glucagon, glybenclamide, glyburide, glyceryl trinitrate, glymepiride, glymepride, granisetron, granulocyte stimulating factor, grepafloxacin, griseofulvin, guanabenz, guanabenz acetate, halofantrine, halofantrine HCl, haloperidol, hydrocortisone, hyoscyamine, ibufenac, ibuprofen, imipenem, indinavir, indivir, indomethacin, insulin, interleukin-3, irbesartan, irinotecan, isosorbide dinitrate, isosorbide mononitrate, isotretinoin, isoxazole, isradipine, itraconazole, ivermectin, ketoconazole, ketoprofen, ketorolac, ketotifen, labetalol, lamivudine, lamotrigine, lanatoside C, lanosprazole, leflunomide, levofloxacin, levothyroxine, lisinopril, lomefloxacin, lomustine, loperamide, loratadine, lorazepam, lorefloxacin, lormetazepam, losartan, lovastatin, L-thryroxine, lysuride, lysuride maleate, maprotiline, maprotiline HCl, mazindol, mebendazole, meclofenamic acid, meclozine, meclozine HCl, medazepam, medigoxin, medroxyprogesterone acetate, mefenamic acid, mefloquine, mefloquine HCl, megesterol acetate, melonicam, melphalan, mepacrine, mepenzolate bromide, meprobamate, meptazinol, mercaptopurine, mesalazine, mesoridazine, mesoridiazine, mestranol, mesylate, metformin, methadone, methaqualone, methoin, methotrexate, methoxsalen, methsuximide, methylphenidate, methylphenobarbital, methylphenobarbitone, methylprednisolone, methyltestosterone, methysergide, methysergide maleate, metoclopramide, metolazone, metoprolol, metronidazole, mianserin, mianserin HCl, miconazole, midazolam, miglitol, minoxidil, mitomycins, mitotane, mitoxantrone, mofetil, molindone, montelukast, morphine, mortriptyline, moxifloxacin, moxifloxacin HCl, mycophenolate, nabumetone, nadolol, nalbuphine, nalidixic acid, naproxen, naratriptan, naratriptan HCl, natamycin, nedocromil sodium, nefazodone, nelfinavir, nerteporfin, neutontin, nevirapine, nicardipine, nicardipine HCl, nicotine, nicoumalone, nifedipine, nilutamide, nimesulide, nimodipine, nimorazole, nisoldipine, nitrazepam, nitro furantoin, nitrofurazone, nizatidine, non-essential fatty acids, norethisterone, norfloxacin, norgestrel, nortriptyline HCl, nystatin, oestradiol, ofloxacin, olanzapine, omeprazole, ondansetron, ondansetron HCL, oprelvekin, omidazole, oxacillin, oxamniquine, oxantel, oxantel embonate, oxaprozin, oxatomide, oxazepam, oxcarbazepine, oxfendazole, oxiconazole, oxprenolol, oxybutynin, oxyphenbutazone, oxyphencylcimine, oxyphencylcimine HCl, paclitaxel, pamidronate, paramethadione, paricalcitol, paroxetine, paroxetine HCl, penicillins, pentaerythritol tetranitrate, pentazocine, pentobarbital, pentobarbitone, pentoxifylline, perchloperazine, perfloxacin, pericyclovir, perphenazine, perphenazine pimozide, phenacemide, phenbenzamine, phenindione, pheniramine, phenobarbital, phenobarbitone, phenoxybenzamine HCl, phensuximide, phentermine, phenylalanine, phenylbutazone, phenytoin, physostigmine, phytonodione, pimozide, pindolol, pioglitazone, piroxicam, pizotifen, pizotifen maleate, pramipexol, pramipexole, pranlukast, pravastatin, praziquantel, prazosin, prazosin HCl, prednisolone, prednisone, pregabalin, primidone, probenecid, probucol, procarbazine, procarbazine HCl, prochlorperazine, progesterone, proguanil, proguanil HCl, propofol, propranolol, propylthiouracil, pseudoephedrine, pyrantel, pyrantel embonate, pyridostigmine, pyrimethamine, quetiapine, quinapril, quinidine, quinidine sulfate, quinine, quinine sulfate, rabeprazole, rabeprazole sodium, raloxifene, raloxifene HCl, ranitidine, ranitidine HCl, recombinant human growth hormone, refocoxib, remifentanil, repaglinide, reserpine, residronate, retinoids, ricobendazole, rifabutin, rifabutine, rifampicin, rifampin, rifapentine, rimantadine, rimexolone, risperodone, ritonavir, rizatriptan, rizatriptan benzoate, robinirole HCl, ropinirole, rosiglitazone, roxatidine, roxithromycin, salbutamol, salmon calcitonin (sCT), saquinavir, selegiline, sertindole, sertraline, sertraline HCl, sibutramine, sibutramine HCl, sildenafil, sildenafil citrate, simvastatin, sirolimus, sodium cefazoline, somatostatin, sparfloxacin, spiramycins, spironolactone, stanozolol, stavudine, stavueline, stiboestrol, sulconazole, sulconazole nitrate, sulfabenzamide, sulfacetamide, sulfadiazine, sulfadoxine, sulfafurazole, sulfamerazine, sulfamethoxazole, sulfapyridine, sulfasalazine, sulindac, sulphabenzamide, sulphacetamide, sulphadiazine, sulphadoxine, sulphafurazole, sulphamerazine, sulphamethoxazole, sulphapyridine, sulphasalazine, sulphin-pyrazone, sulpiride, sulthiame, sumatriptan, sumatriptan succinate, tacrine, tacrolimus, tamoxifen, tamoxifen citrate, tamsulosin, tamsulosin HCl, targretin, tazarotene, telmisartan, temazepam, teniposide, terazosin, terazosin HCl, terbinafine HCl, terbutaline, terbutaline sulfate, terconazole, terenadine, terfenadine, testolactone, testosterone, tetracycline, tetrahydrocannabinol, tetramisole, thiabendazole, thioguanine, thioridazine, tiagabine, tiagabine HCl, tibolone, ticlidopine, ticlopidine, tiludronate, timolol, tinidazole, tioconazole, tirofibran, tizanidine, tizanidine HCl, tolazamide, tolbutamide, tolcapone, tolmetin, tolterodine, topiramate, topotecan, topotecan HCl, toremifene, toremifene citrate, tramadol, trazodone, trazodone HCl, tretinoin, triamcinolone, triamterene, triazolam, trifluoperazine, trimethoprim, trimipramine, trimipramine maleate, troglitazone, tromethamine, tropicamide, trovafloxacin, tumor necrosisi factor, undecenoic acid, ursodeoxycholic acid, valacylcovir, valproic acid, valsartan, vancomycin, vasopressin, venlafaxine HCl, verteporfin, vigabatrin, vinblastine, vincristine, vinorelbine, vitamin A, vitamin B₂, vitamin D, vitamin E and vitamin K, vitamin K₅, vitamin K₆, vitamin K₇, vitamin K-S (II), zafirlukast, zileuton, zolmitriptan, zolpidem, and zopiclone.

Oil-soluble vitamins, e.g., vitamin E or compounds having vitamin E activity, are particularly preferred components of the compositions and dosage forms described herein. Examples of compounds having vitamin E activity include, but are not limited to, α-tocopherol, β-tocopherol, γ-tocopherol, δ-tocopherol, α-tocotrienol, β-tocotrienol, γ-tocotrienol, δ-tocotrienol, α-tocopherol acetate, β-tocopherol acetate, γ-tocopherol acetate, δ-tocopherol acetate, α-tocotrienol acetate, β-tocotrienol acetate, γ-tocotrienol acetate, δ-tocotrienol acetate, α-tocopherol succinate, β-tocopherol succinate, γ-tocopherol succinate, δ-tocopherol succinate, α-tocotrienol succinate, β-tocotrienol succinate, γ-tocotrienol succinate, δ-tocotrienol succinate, and mixtures thereof.

However, it should be noted that oil-soluble vitamins, particularly vitamin E and compounds having vitamin E activity, e.g., antioxidant activity, may also serve as a component of the carrier for improving the delivery of one or more therapeutic agents in the present compositions. Oil-soluble vitamins, particularly vitamin E and compounds having vitamin E activity, may improve the solubilization of other therapeutic agents in the composition and/or in an aqueous dispersion of the composition when placed in an aqueous medium. Furthermore, the stability of the therapeutic agent(s) in the composition and/or the stability of the composition as a whole may be enhanced when oil-soluble vitamins are present in the formulation. Additionally, the extent and/or A consistency of absorption of the therapeutic agent(s) may be improved in the presence of the oil-soluble vitamin, particularly vitamin E and compounds having vitamin E activity, due to improved solubilization of the therapeutic agent and/or improved permeability of the therapeutic agent across an absorption barrier, e.g., the mucosal membrane at the site of absorption. It should also be noted that the oil-soluble vitamins may be simply regarded as part of the carrier, rather than as therapeutic agents, if another therapeutic agent is present in the pharmaceutical composition (and intended for a therapeutic purpose unrelated to any therapeutic activity of the oil-soluble vitamin).

The active agent in the present dosage forms may be an integral part of the composition, or it may be present in a coating on the dosage form, e.g., on a capsule, tablet, or caplet, or on each of a plurality of granules, beads, or pellets. In preferred embodiments, the active agent, e.g., low molecular weight heparin, is present as a part of the coating on the dosage form. Alternatively, the active agent is present as an integral part of the composition and is at least partially solubilized or suspended therein. The active agent may take any number of physical forms, e.g., it may be in crystalline, amorphous, nanosized, micronized or milled form.

It will be recognized that the compositions and methods described herein may include a single therapeutic agent or a combination of two or more therapeutic agents. Such combinations provide improved patient compliance and/or a superior safety and efficacy profile when compared to the individual administration of single therapeutic agents. Although conventional doses of each therapeutic agent in the combination may be used, the combination may allow for dose modification, either higher or lower, given the improved patient compliance or superior safety and efficacy profile. Such modifications to the dose are based upon the overall therapeutic benefit to the patient, which can be evaluated by one of ordinary skill in the art using routine methodology. For example, lower doses of many therapeutic agents can still provide significant therapeutic benefits while decreasing potential side effects.

It may be desirable to include one or more additional active agents in the dosage forms herein. A wide range of additional active agents may be co-administered with the hydrophilic drug, including both hydrophilic and lipophilic active agents, particularly although not necessarily agents that potentiate certain effects of the hydrophilic drug, or vice versa. For example, co-administration with aspirin would be desirable to treat unstable angina, and co-administration with warfarin would be indicated for prophylaxis of deep-vein thrombosis.

Of course, salts, metabolic precursors, derivatives and mixtures of any of the therapeutic agents listed herein may also be used where desired.

### 4. Concentrations

The components of the pharmaceutical compositions of the present invention in amounts such that upon dilution with an aqueous medium, the composition forms a clear, aqueous dispersion. The determining concentrations of components to form clear aqueous dispersions are the concentrations of triglyceride and surfactants, with the amount of the therapeutic agent, if present, being chosen as described below. The relative amounts of triglycerides and surfactants are readily determined by observing the properties of the resultant dispersion; i.e., when the relative amounts of these components are within a suitable range, the resultant aqueous dispersion is optically clear. When the relative amounts are outside the suitable range, the resulting dispersion is visibly “cloudy,” resembling a conventional emulsion or multiple-phase system. Although a visibly cloudy solution may be potentially useful for some applications, such a system would suffer from many of the same disadvantages as conventional prior art formulations, as described above.

The aqueous medium can comprise body fluids naturally occurring in the subject to whom the pharmaceutical compositions are administered. Such naturally occurring fluids can be the fluids occurring or produced in the oral cavity, nasal cavity, respiratory system, digestive system, for example, gastric juice, intestinal fluid, saliva, and lung fluid. The aqueous medium can also be fluids simulating such naturally occurring body fluids, for example, simulated gastric fluid and simulated intestinal fluid in absence or presence of variable amount of naturally occurring, semi-synthetic, or synthetic surface active materials. Typical surface active materials include proteins such as pepsin and pancreatin (which also possess enzymatic activity), bile acids, bile salts, phospholipids such as lecithins and lysolecithins and synthetic surfactant such as Tweens, sodium lauryl sulfate, etc. The concentration of such materials present in the simulated fluids can be in the range of about 0.01 wt. % to about 10 wt. %, most typically in the range of about 0.01 wt. % to about 1 wt. %. Occasionally, other organic materials such glycerol, alcohol, and polymers such as PEG and PVP can be incorporated in the simulated fluids to adjust properties such as viscosity, osmolarity, and dielectric constant; such materials can also serve as solubilizing agents.

A convenient method of determining the appropriate relative concentrations for any particular triglyceride is as follows. A convenient working amount of a hydrophilic surfactant is provided, and a known amount of the triglyceride is added. The mixture is stirred, with the aid of gentle heating if desired, then is diluted with purified water to prepare an aqueous dispersion. Any dilution amount can be chosen, but convenient dilutions are those within the range expected in vivo, about a 10 to 250-fold dilution. In the Examples herein, a convenient dilution of 100-fold was chosen. The aqueous dispersion is then assessed qualitatively for optical clarity. The procedure can be repeated with incremental variations in the relative amount of triglyceride added, to determine the maximum relative amount of triglyceride that can be present to form a clear aqueous dispersion with a given hydrophilic surfactant, i.e., when the relative amount of triglyceride is too great, a hazy or cloudy dispersion is formed.

The amount of triglyceride that can be solubilized in a clear aqueous dispersion is increased by repeating the above procedure, but substituting a second hydrophilic surfactant, or a hydrophobic surfactant, for part of the originally-used hydrophilic surfactant, thus keeping the total surfactant concentration constant. Of course, this procedure is merely exemplary, and the amounts of the components can be chosen using other methods, as desired.

It has been surprisingly found that mixtures of surfactants including two surfactants can solubilize a greater relative amount of triglyceride than a single surfactant. Similarly, mixtures of surfactants including a hydrophilic surfactant and a hydrophobic surfactant can solubilize a greater relative amount of triglyceride than either surfactant by itself. It is particularly surprising that when the surfactant mixture includes a hydrophilic surfactant and a hydrophobic surfactant, the solubility of the triglyceride is greater than, for example, in the hydrophilic surfactant itself. Furthermore, a greater amount of the hydrophobic surfactant can be solubilized when a triglyceride is present for a given amount of a hydrophilic surfactant. Thus, contrary to conventional knowledge in the art, the total amount of water-insoluble component (triglyceride plus hydrophobic surfactant) exceeds the amount of hydrophobic surfactant or triglyceride that can be solubilized by the same amount of hydrophilic surfactant. This unexpected finding shows a surprising and non-intuitive relationship between the hydrophilic and hydrophobic components.

It should be emphasized that the optical clarity is determined in the diluted composition (the aqueous dispersion), and not in the pre-concentrate. Thus, for example, U.S. Pat. No. 4,719,239 shows optically clear compositions containing water, oil, and a 3:7 mixture of PEG-glycerol monooleate and caprylic-capric acid glycerol esters, but the compositions contain no more that about 75% by weight water, or a dilution of the pre-concentrate of no more than 3 to 1. Upon dilution with water in a ratio of more than about 3 to 1, the compositions of the cited reference phase-separate into multi-phase systems, as is shown, for example, in the phase diagram of FIG. 2 in the '239 patent. In contrast, the compositions of the present invention, when diluted to values typical of dilutions encountered in vivo, or when diluted in vivo upon administration to a patient, remain as clear aqueous dispersions. Thus, the clear aqueous dispersions of the present invention are formed upon dilution of about 10 to about 250-fold or more.

As an alternative to qualitative visual assessment of optical clarity, the optical clarity of the aqueous dispersion can be measured using standard quantitative techniques for turbidity assessment. One convenient procedure to measure turbidity is to measure the amount of light of a given wavelength transmitted by the solution, using, for example, a UV-visible spectrophotometer. Using this measure, optical clarity corresponds to high transmittance, since cloudier solutions will scatter more of the incident radiation, resulting in lower transmittance measurements. If this procedure is used, care should be taken to insure that the composition itself does not absorb light of the chosen wavelength, as any true absorbance necessarily reduces the amount of transmitted light and falsely increases the quantitative turbidity value. In the absence of chromophores at the chosen wavelength, suitable dispersions at a dilution of 100× should have an apparent absorbance of less than about 0.3, preferably less than about 0.2, and more preferably less than about 0.1.

Other methods of characterizing optical clarity, such as direct particle size measurement and other methods known in the art may also be used.

It should be emphasized that any or all of the available methods may be used to ensure that the resulting aqueous dispersions possess the requisite optical clarity. For convenience, however, the present inventors prefer to use the simple qualitative procedure; i.e., simple visible observation. However, in order to more fully illustrate the practice of the present invention, both qualitative observation and spectroscopic measures are used to assess the dispersion clarity in the Examples herein.

If present, the therapeutic agent is solubilized in the carrier, e.g., the triglyceride, the surfactant(s), or both the triglyceride and the surfactant(s). Alternatively, the therapeutic agent can be solubilized in the aqueous medium used to dilute the preconcentrate to form an aqueous dispersion. The maximum amount of therapeutic agent that can be solubilized is readily determined by simple mixing, as the presence of any non-solubilized therapeutic agent is apparent upon visual examination.

In one embodiment, the therapeutic agent is present in an amount up to the maximum amount that can be solubilized in the composition. In another embodiment, the therapeutic agent is present in a first amount that is solubilized, and a second amount that remains unsolubilized but dispersed. This may be desirable when, for example, a larger dose of the therapeutic agent is desired. Although not all of the therapeutic agent is solubilized, such a composition presents advantages over conventional compositions, since at least a portion of the therapeutic agent is present in the clear aqueous dispersion phase. Of course, in this embodiment, the optical clarity of the resultant aqueous dispersion is determined before the second non-solubilized amount of the therapeutic agent is added.

In some contexts, the compositions will be “substantially free of water.” “Substantially free of water” as used herein is intended to mean that the composition or dosage form contains less than 20% water (v/v). More preferably, the composition or dosage form contains less than about 10% water and most preferably less than about 5% water. In turn, this means that any water present will not form a continuous aqueous phase.

Other considerations well known to those skilled in the art will further inform the choice of specific proportions of components, e.g., surfactants and triglycerides, of the compositions. These considerations include the degree of bioacceptability of the compounds, and the desired dosage of therapeutic agent to be provided. In some cases, the amount of triglyceride or therapeutic agent actually used in a pharmaceutical composition according to the present invention will be less than the maximum that can be solubilized, and it should be apparent that such compositions are also within the scope of the present invention.

### 5. Solubilizers

If desired, the pharmaceutical compositions of the present invention can optionally include additional compounds to enhance the solubility of the therapeutic agent or the triglyceride in the composition. Examples of such compounds, referred to as “solubilizers,” include:

alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulosic polymers, cyclodextrins and cyclodextrin derivatives;

ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide);

amides, such as 2-pyrrolidone, 2-piperidone, ∈-caprolactam, N-alkylpyrrolidone, N-hydroxyalkylpyrrolidone, N-alkylpiperidone, N-alkylcaprolactam, dimethylacetamide, and polyvinylpyrrolidone;

esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof;

and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), and water.

One type of solubilizer that may be used is a vitamin E substance. This group of solubilizers includes a substance belonging to the group of α-, β-, γ-, δ-, ζ1-, ζ2- and ∈-tocopherols, their dl, d and l forms and their structural analogues, such as tocotrienols;—which includes substances with the tocol structure [2-methyl-2-(4,8,12-trimethyltridecyl)chroman-6-ol] or the tocotrienol structure [2-methyl-2-(4,8,12-trimethyltrideca-3,7,11-trienyl)chroman-6-o-l], in particular the all trans-(E,E) tocotrienols. Particularly preferred vitamin E substances include the mono-, di-, trimethyl-tocol derivatives, commonly known as tocopherols, such as α-tocopherol [5,7,8-trimethyl-], β-tocopherol [5,8-dimethyl-], γ-tocopherol [7,8-dimethyl], ζ2-tocopherol [5,7-dimethyl-], δ-tocopherol [8-methyl-], q-tocopherol [7-methyl]; and the corresponding mono-, di-, and trimethyltoctrienol derivatives, commonly known as tocotrienols, such as α-tocotrienol (or ζ1-tocopherol) [5,7,8-trimethyl-], β-tocotrienol (or ∈-tocopherol) [5,8-dimethyl], γ-tocotrienol [7,8-dimethyl], and 8-tocotrienol [8-methyl-]. Included are their mixed racemic dl-forms, the pure d- and l-enantiomers and the corresponding derivatives, e.g., esters, produced with organic acids; and mixtures thereof.

One skilled in the art can easily identify vitamin E substances that may serve as effective solubilizers by, for example, mixing a particular vitamin E substance with fenofibrate and determining the extent of solubility. Preferred vitamin E substances for use in the present invention include tocopherols, tocotrienols and tocopherol derivatives with organic acids such as acetic acid, propionic acid, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid, polyethylene glycol succinate and salicylic acid. Particularly preferred vitamin E substances include alpha-tocopherol, alpha-tocopherol acetate, alpha-tocopherol acid succinate, alpha-tocopherol polyethylene glycol succinate and mixtures thereof.

Another group of solubilizers are monohydric alcohol esters of organic acids. The monohydric alcohol can be, for example, ethanol, isopropanol, t-butanol, a fatty alcohol, phenol, cresol, benzyl alcohol or a cycloalkyl alcohol. The organic acid can be, for example, acetic acid, propionic acid, butyric acid, a fatty acid of 6-22 carbon atoms, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid and salicylic acid. Preferred solubilizers in this group include trialkyl citrates, lower alcohol fatty acid esters and lactones. Preferred trialkyl citrates include triethyl citrate, acetyltriethyl citrate, tributyl citrate, acetyltributyl citrate and mixtures thereof with triethyl citrate being particularly preferred. Lower alcohol fatty acid esters, as the name implies, comprise a lower alcohol moiety, i.e., containing 2-4 carbon atoms, and a fatty acid moiety of about 6-22 carbon atoms. Particularly preferred lower alcohol fatty acid esters include ethyl oleate, ethyl linoleate, ethyl caprylate, ethyl caprate, isopropyl myristate, isopropyl palmitate and mixtures thereof. Lactones may also serve as a solubilizer. Examples include ∈-caprolactone, δ-valerolactone, β-butyrolactone, isomers thereof and mixtures thereof.

The solubilizer may be a nitrogen-containing solvent. Preferred nitrogen-containing solvents include dimethylformamide, dimethylacetamide, N-alkylpyrrolidone, N-hydroxyalkylpyrrolidone, N-alkylpiperidone, N-alkylcaprolactam and mixtures thereof wherein alkyl is a C₁₋₁₂ branched or straight chain alkyl. Particularly preferred nitrogen-containing solvents include N-methyl 2-pyrrolidone, N-ethyl 2-pyrrolidone or a mixture thereof. Alternatively, the nitrogen-containing solvent may be in the form of a polymer such as polyvinylpyrrolidone.

Another group of solubilizers includes phospholipids. Preferred phospholipids include phosphatidylcholine, phosphatidylethanolamine, phosphatidylserine, phosphatidylinositol, lecithins, lysolecithins, lysophosphatidylcholine, polyethylene glycolated phospholipids/lysophospholipids, lecithins/lysolecithins and mixtures thereof.

Another group of preferred solubilizers are glycerol acetates and acetylated glycerol fatty acid esters. Preferred glycerol acetates include acetin, diacetin, triacetin and mixtures thereof, with triacetin being particularly preferred. Preferred acetylated glycerol fatty acid esters include acetylated monoglycerides, acetylated diglycerides and mixtures thereof. In a most preferred embodiment, the acetylated monoglyceride is a distilled acetylated monoglyceride.

In addition, the solubilizer may be a glycerol fatty acid ester. The fatty acid component is about 6-22 carbon atoms. The glycerol fatty acid ester can be a monoglyceride, diglyceride, triglyceride or mixtures thereof. Preferred glycerol fatty acid esters include monoglycerides, diglycerides, medium chain triglycerides with fatty acids having about 6-12 carbons and mixtures thereof. Particularly preferred glycerol fatty acid esters include medium chain monoglycerides with fatty acids having about 6-12 carbons, medium chain diglycerides with fatty acids having about 6-12 carbons and mixtures thereof.

The solubilizer may be a propylene glycol ester. Preferred propylene glycol esters include propylene carbonate, propylene glycol monoacetate, propylene glycol diacetate, propylene glycol fatty acid esters, acetylated propylene glycol fatty acid esters and mixtures thereof. Alternatively, the propylene glycol fatty acid ester may be a propylene glycol fatty acid monoester, propylene glycol fatty acid diester or mixture thereof. The fatty acid has about 6-22 carbon atoms. It is particularly preferred that the propylene glycol ester is propylene glycol monocaprylate. Other preferred propylene glycol esters include propylene glycol dicaprylate, propylene glycol dicaprate, propylene glycol dicaprylate/dicaprate and mixtures thereof.

Another group of solubilizers are ethylene glycol esters. Ethylene glycol esters include monoethylene glycol monoacetates, diethylene glycol esters, polyethylene glycol esters and mixtures thereof. Additional examples include ethylene glycol monoacetates, ethylene glycol diacetates, ethylene glycol fatty acid monoesters, ethylene glycol fatty acid diesters, and mixtures thereof. Alternatively, the ethylene glycol ester may be a polyethylene glycol fatty acid monoesters, polyethylene glycol fatty acid diesters or mixtures thereof. Again, the fatty acid component will contain about 6-22 carbon atoms. Particularly preferred ethylene glycol esters are those obtained from the transesterification of polyethylene glycol with a triglyceride or a vegetable oil or mixture thereof and include, for example, those marketed under the Labrafil® and Labrasol® names.

Other solubilizers that may be used in the present invention are disclosed in U.S. Pat. Nos. 6,982,281 and 6,761,903, both to Chen et al. Preferred solubilizers that are not vitamin E substances for use in the present invention include fatty acid esters of glycerol, acetylated mono- and diglycerides, fatty acid esters of propylene glycol, trialkyl citrate, glycerol acetate, and lower alcohol fatty acid esters.

Mixtures of solubilizers are also within the scope of the invention. Except as indicated, these compounds are readily available from standard commercial sources.

Preferred solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-600, glycofurol, transcutol, propylene glycol, and dimethyl isosorbide. Particularly preferred solubilizers include sorbitol, glycerol, triacetin, ethyl alcohol, PEG-400, glycofuirol and propylene glycol.

The amount of solubilizer that can be included in compositions of the present invention is not particularly limited. Of course, when such compositions are ultimately administered to a patient, the amount of a given solubilizer is limited to a bioacceptable amount, which is readily determined by one of skill in the art. In some circumstances, it may be advantageous to include amounts of solubilizers far in excess of bioacceptable amounts, for example, to maximize the concentration of therapeutic agent, with excess solubilizer removed prior to providing the composition to a patient using conventional techniques, such as distillation or evaporation. Thus, if present, the solubilizer can be in a concentration of 50%, 100%, 200%, or up to about 400% by weight, based on the amount of surfactant. If desired, very small amounts of solubilizers may also be used, such as 25%, 10%, 5%, 1% or even less. Typically, the solubilizer will be present in an amount of about 1% to about 100%, more typically about 5% to about 25% by weight or about 10% to about 25% by weight.

### 6. Enzyme Inhibitors

When the therapeutic agent is subject to enzymatic degradation, the compositions can include an enzyme-inhibiting agent. Enzyme inhibiting agents are shown for example, in Bemskop-Schnurch, A., “The use of inhibitory agents to overcome enzymatic barrier to perorally administered therapeutic peptides and proteins”, J Controlled Release 52, 1-16 (1998), the disclosure of which is incorporated herein by reference.

Generally, inhibitory agents can be divided into the following classes:

Inhibitors that are not based on amino acids, such as P-aminobenzamidine, FK-448, camostat mesylate, sodium glycocholate;

Amino acids and modified amino acids, such as aminoboronic acid derivatives and n-acetylcysteine;

Peptides and modified peptides, such as bacitracin, phosphinic acid dipeptide derivatives, pepstatin, antipain, leupeptin, chymostatin, elastatin, bestatin, hosphoramindon, puromycin, cytochalasin potatocarboxy peptidase inhibitor, and amastatin;

Polypeptide protese inhibitors, such as aprotinin (bovine pancreatic trypsin inhibitor), Bowman-Birk inhibitor and soybean trypsin inhibitor, chicken egg white trypsin inhibitor, chicken ovoinhibitor, and human pancreatic trypsin inhibitor;

Complexing agents, such as EDTA, EGTA, 1,10-phenanthroline and hydroxychinoline; and

Mucoadhesive polymers and polymer-inhibitor conjugates, such as polyacrylate derivatives, chitosan, cellulosics, chitosan-EDTA, chitosan-EDTA-antipain, polyacrylic acid-bacitracin, carboxymethyl cellulose-pepstatin, polyacrylic acid-Bwoman-Birk inhibitor.

The choice and levels of the enzyme inhibitor are based on toxicity, specificity of the proteases and the potency of the inhibition. The inhibitor can be suspended or solubilized in the composition preconcentrate, or added to the aqueous diluent or as a beverage.

It is contemplated that an inhibitor can function solely or in combination as:

a competitive inhibitor, by binding at the substrate binding site of the enzyme, thereby preventing the access to the substrate; examples of inhibitors believed to operate by this mechanism are antipain, elastatinal and the Bowman Birk inhibitor;

a non-competitive inhibitor which can be simultaneously bound to the enzyme site along with the substrate, as their binding sites are not identical; and/or a complexing agent due to loss in enzymatic activity caused by deprivation of essential metal ions out of the enzyme structure.

### 7. Other Additives

Other additives conventionally used in pharmaceutical compositions can be included, and these additives are well known in the art. Such additives include detackifiers, anti-foaming agents, buffering agents, antioxidants, preservatives, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired.

### 8. Dosage Forms

The pharmaceutical compositions of the present invention can be formulated as a preconcentrate in a liquid, semi-solid, or solid form, or as an aqueous or organic diluted preconcentrate. In the diluted form, the diluent can be water, an aqueous medium, a buffer, an organic solvent, a beverage, a juice, or mixtures thereof. If desired, the diluent can include components soluble therein, such as a therapeutic agent, an enzyme inhibitor, solubilizers, additives, and the like.

The compositions can be processed according to conventional processes known to those skilled in the art, such as lyophilization, encapsulation, compression, melting, extrusion, drying, chilling, molding, spraying, coating, comminution, mixing, homogenization, sonication and granulation, to produce the desired dosage form.

The dosage form is not particularly limited. Thus, compositions of the present invention can be formulated as pills, capsules, caplets, tablets, granules, beads or powders. Granules, beads and powders can, of course, be further processed to form pills, capsules, caplets or tablets. When formulated as a capsule, the capsule can be a hard or soft gelatin capsule, a starch capsule, or a cellulosic capsule. Such dosage forms can further be coated with, for example, a seal coating or an enteric coating. The term “enteric coated capsule” as used herein means a capsule coated with a coating resistant to acid; i.e., an acid resistant enteric coating. Enteric coated compositions of this invention protect therapeutic peptides or proteins in a restricted area of drug liberation and absorption, and reduce or even exclude extensive dilution effects. Although solubilizers are typically used to enhance the solubility of a hydrophobic therapeutic agent, they may also render the compositions more suitable for encapsulation in hard or soft gelatin capsules. Thus, the use of a solubilizer such as those described above is particularly preferred in capsule dosage forms of the pharmaceutical compositions. If present, these solubilizers should be added in amounts sufficient to impart to the compositions the desired solubility enhancement or encapsulation properties.

Although formulations specifically suited to oral administration are presently preferred, the compositions of the present invention can also be formulated for topical, transdermal, buccal, ocular, pulmonary, vaginal, rectal, transmucosal or parenteral administration, as well as for oral administration. Thus, the dosage form can be a solution, suspension, emulsion, cream, ointment, lotion, suppository, spray, aerosol, paste, gel, drops, douche, ovule, wafer, troche, cachet, syrup, elixir, or other dosage form, as desired. If formulated as a suspension, the composition can further be processed in capsule form.

When formulated as a sprayable solution or dispersion, a dosage form of a multiparticulate carrier coated onto a substrate with the pharmaceutical compositions described herein can be used. The substrate can be a granule, a particle, or a bead, for example, and formed of a therapeutic agent or a pharmaceutically acceptable material. The multiparticulate carrier can be enteric coated with a pharmaceutically acceptable material as is well known to those skilled in the art.

Other additives may be included, such as are well known in the art, to impart the desired consistency and other properties to the formulation.

### 9. Specific Embodiments

In all of the embodiments described herein, the triglyceride and surfactants are present in amounts such that upon mixing with an aqueous medium, either in vitro or in vivo, a clear, aqueous dispersion is formed. This optical clarity in an aqueous dispersion defines the appropriate relative concentrations of the triglyceride and surfactant components, but does not restrict the dosage form of the compositions to an aqueous dispersion, nor does it limit the compositions of the invention to optically clear dosage forms. Thus, the appropriate concentrations of the triglyceride and surfactants are determined by the optical clarity of a dispersion formed by the composition preconcentrate and an aqueous medium in a dilution of about 10 to about 250-fold, as a preliminary matter. Once the appropriate concentrations are determined, the pharmaceutical compositions can be formulated as described in the preceding section, without regard to the optical clarity of the ultimate formulation. Of course, optically clear aqueous dispersions, and their preconcentrates, are preferred formulations. In one embodiment, the present invention relates to pharmaceutical compositions having a triglyceride and a carrier, the carrier including at least two surfactants, at least one of which is hydrophilic. The triglyceride and surfactants are present in amounts such that upon mixing with an aqueous medium, either in vitro or in vivo, the composition forms a clear aqueous dispersion. In a particular aspect of this embodiment, the composition can contain more triglyceride than can be solubilized in a clear aqueous dispersion having only one surfactant, the surfactant being hydrophilic. Thus, this embodiment provides a higher concentration of triglyceride than is achievable with a single hydrophilic surfactant, resulting in a reduced triglyceride to hydrophilic surfactant ratio and enhanced biocompatibility.

In another embodiment, the present invention relates to pharmaceutical compositions having a triglyceride and a carrier, the carrier including at least one hydrophilic surfactant and at least one hydrophobic surfactant. The triglyceride and surfactants are present in amounts such that upon mixing with an aqueous medium, either in vitro or in vivo, the composition forms a clear aqueous dispersion. In a particular aspect of this embodiment, the composition contains more triglyceride than can be solubilized in a clear aqueous dispersion having a hydrophilic surfactant but not having a hydrophobic surfactant.

In another embodiment, the triglyceride itself can have therapeutic value as, for example, a nutritional oil, or absorption-promoting value as, for example, a long-chain triglyceride (LCT) or a medium-chain triglyceride (MCT). Thus, in this embodiment, the present invention provides pharmaceutical compositions including a triglyceride having nutritional and/or absorption-promoting value, and a carrier. The carrier includes at least two surfactants, at least one of which is hydrophilic. Optionally, the carrier can include at least one hydrophilic surfactant and at least one hydrophobic surfactant. The triglyceride and surfactants are present in amounts such that upon dilution with an aqueous medium, either in vitro or in vivo, the composition forms a clear aqueous dispersion.

In another embodiment, the present invention relates to a pharmaceutical composition that includes a therapeutic agent, a triglyceride and a carrier. The carrier includes at least two surfactants, at least one of which is hydrophilic. Optionally, the carrier includes at least one hydrophilic surfactant and at least one hydrophobic surfactant. The triglyceride, and surfactants are present in amounts such that upon dilution with an aqueous medium, either in vitro or in vivo, the composition forms a clear aqueous dispersion. The therapeutic agent is present in two amounts, a first amount of the therapeutic agent solubilized in the clear aqueous dispersion, and a second amount of the therapeutic agent that remains non-solubilized but dispersed.

In another aspect, the present invention relates to triglyceride-containing pharmaceutical compositions as described in the preceding embodiments, which further include a therapeutic agent. In particular embodiments, the therapeutic agent is a hydrophobic drug or a hydrophilic drug. In other embodiments, the therapeutic agent is a nutritional agent. In still further embodiments, the therapeutic agent is a cosmeceutical agent.

### 10. Preparation of Pharmaceutical Compositions

### A. Overview

The pharmaceutical compositions of the present invention can be prepared by conventional methods well known to those skilled in the art. Of course, the specific method of preparation will depend upon the ultimate dosage form. For dosage forms substantially free of water, i.e., when the composition is provided in a pre-concentrate form for later dispersion in vitro or in vivo in an aqueous system, the composition is prepared by simple mixing of the components to form a pre-concentrate. The mixing process can be aided by gentle heating, if desired. For compositions in the form of an aqueous dispersion, the pre-concentrate form is prepared, then the appropriate amount of an aqueous medium is added. Upon gentle mixing, a clear aqueous dispersion is formed. If any water-soluble enzyme inhibitors or additives are included, these may be added first as part of the pre-concentrate, or added later to the clear aqueous dispersion, as desired. The compositions can be prepared with or without a therapeutic agent, and a therapeutic agent may also be provided in the diluent, if desired.

As previously noted, in another embodiment, the present invention includes a multi-phase dispersion containing a therapeutic agent. In this embodiment, a pharmaceutical composition includes a triglyceride and a carrier, which forms a clear aqueous dispersion upon mixing with an aqueous medium, and an additional amount of non-solubilized therapeutic agent. Thus, the term “multi-phase” as used herein to describe these compositions of the present invention means a composition which when mixed with an aqueous medium forms a clear aqueous phase and a particulate dispersion phase. The carrier and triglycerides are as described above, and can include any of the surfactants, therapeutic agents, solubilizers and additives previously described. An additional amount of therapeutic agent is included in the composition. This additional amount is not solubilized by the carrier, and upon mixing with an aqueous system is present as a separate dispersion phase. The additional amount is optionally a milled, micronized, or precipitated form. Thus, upon dilution, the composition contains two phases: a clear aqueous dispersion of the triglyceride and surfactants containing a first, solubilized amount of the therapeutic agent, and a second, non-solubilized amount of the therapeutic agent dispersed therein. It should be emphasized that the resultant multi-phase dispersion will not have the optical clarity of a dispersion in which the therapeutic agent is fully solubilized, but will appear to be cloudy, due to the presence of the non-solubilized phase. Such a formulation may be useful, for example, when the desired dosage of a therapeutic agent exceeds that which can be solubilized in the carrier and/or triglyceride. The formulation may also contain additives, as described above.

One skilled in the art will appreciate that a therapeutic agent may have a greater solubility in the pre-concentrate composition than in the aqueous dispersion, so that meta-stable, supersaturated solutions having apparent optical clarity but containing a therapeutic agent in an amount in excess of its solubility in the aqueous dispersion can be formed. Such super-saturated solutions, whether characterized as clear aqueous dispersions (as initially formed) or as multi-phase solutions (as would be expected if the meta-stable state breaks down), are also within the scope of the present invention.

The multi-phase formulation can be prepared by the methods described above. A pre-concentrate is prepared by simple mixing of the components, with the aid of gentle heating, if desired. It is convenient to consider the therapeutic agent as divided into two portions, a first solubilizable portion that will be solubilized and contained within the clear aqueous dispersion upon dilution, and a second non-solubilizable portion that will remain non-solubilized. When the ultimate dosage form is non-aqueous, the first and second portions of the therapeutic agent are both included in the pre-concentrate mixture. When the ultimate dosage form is aqueous, the composition can be prepared in the same manner, and upon dilution in an aqueous system, the composition will form the two phases as described above, with the second non-solubilizable portion of the therapeutic agent dispersed or suspended in the aqueous system, and the first solubilizable portion of the therapeutic agent solubilized in the composition. Alternatively, when the ultimate dosage form is aqueous, the pre-concentrate can be prepared including only the first, solubilizable portion of the therapeutic agent. This pre-concentrate can then be diluted in an aqueous system to form a clear aqueous dispersion, to which is then added the second, non-solubilizable portion of the therapeutic agent to form a multi-phase aqueous composition.

### B. Methods

In another embodiment, the present invention relates to methods of increasing the solubilization of a therapeutic agent in a composition, by providing the therapeutic agent in a composition of the present invention. The composition can be any of the compositions described herein, with or without a therapeutic agent. It is surprisingly found that by using the combinations of triglycerides and surfactants described herein, greater amounts of triglycerides can be solubilized, without resort to unacceptably high concentrations of hydrophilic surfactants.

In another embodiment, the present invention relates to methods of increasing the rate and/or extent of absorption of therapeutic agents by administering to a patient a pharmaceutical composition of the present invention. In this embodiment, the therapeutic agent can be present in the pharmaceutical composition pre-concentrate, in the diluent, or in a second pharmaceutical composition, such as a conventional commercial formulation, which is co-administered with a pharmaceutical composition of the present invention. For example, the delivery of therapeutic agents in conventional pharmaceutical compositions can be improved by co-administering a pharmaceutical composition of the present invention with a conventional composition.

Administration of the compositions and dosage forms described herein may be used to treat any disorder, condition or disease for which the particular therapeutic agent is generally indicated. Dosage regimens and daily dosage for a therapeutic agent can vary a great deal, as a number of factors are involved, including the particular derivative of the therapeutic agent, the age and general condition of the patient, the particular condition or disorder and its severity, and the like. Clearly, however, it is necessary that the dosage given be sufficient to provide the desired pharmacological activity in a patient's circulation.

### C. Characteristics of the Pharmaceutical Compositions

The pharmaceutical compositions or the aqueous dispersions formed upon dilution of the pharmaceutical compositions described herein will generally have the following characteristics:

Rapid formation: upon dilution with an aqueous medium, the composition forms a clear dispersion very rapidly; i.e., the clear dispersion appears to form instantaneously.

Optical clarity: the dispersions are essentially optically clear to the naked eye, and show no readily observable signs of heterogeneity, such as turbidity or cloudiness. More quantitatively, dispersions of the pharmaceutical compositions of the present invention have absorbances (400 nm) of less than about 0.3, and often less than about 0.1, at 100× dilution, as described more fully in the Examples herein. In the multi-phase embodiment of the compositions described herein, it should be appreciated that the optical clarity of the aqueous phase will be obscured by the dispersed particulate non-solubilized therapeutic agent.

Robustness to dilution: the dispersions are surprisingly stable to dilution in aqueous medium. The hydrophobic therapeutic agent remains solubilized for at least the period of time relevant for absorption.

As discussed above, conventional triglyceride-containing formulations suffer the disadvantage that bioabsorption of the therapeutic agents contained therein is dependent upon enzymatic degradation (lipolysis) of the triglyceride components. The solubilization of the triglyceride in an aqueous medium is usually limited if only a hydrophilic surfactant is used to disperse the triglyceride, as is conventional. Without a sufficiently high concentration of the hydrophilic surfactant, an emulsion or milky suspension of the triglyceride is formed, and the triglyceride is present in the form of relatively large oil droplets. In this case, the large size of the triglyceride particles impedes the transport and absorption of the triglyceride or therapeutic agent solubilized in the triglyceride or in the carrier. In addition, the large, thermodynamically unstable triglyceride particles could further impose a risk when the compositions are administered intravenously, by plugging the blood capillaries.

To achieve a high level of fully-solubilized triglyceride would require an amount of the hydrophilic surfactant exceeding that which would be bioacceptable. The pharmaceutical compositions of the present invention, however, solve these and other problems of the prior art by adding a third component, a hydrophobic surfactant or a second hydrophilic surfactant. The solubilization of the triglyceride in the aqueous system is thereby unexpectedly enhanced. Conversely, it is also true that solubilization of a hydrophobic surfactant or a second hydrophilic surfactant is unexpected enhanced based on the presence of the triglyceride in the formulation. Of course, the relative amounts of the hydrophobic surfactant or the second hydrophilic surfactant in the formulation will depend on the type of formulation that is desired, actual components used, nature of the therapeutic agent, and so forth. These and other factors are routinely considered by those of skill in the art in determining the amount of each component to be added to a formulation. In addition, a suitable amount of each component to form the desired formulations can be readily determined by routine experimentation.

It is also unexpectedly found that the total amount of solubilized water-insoluble components, the triglyceride and hydrophobic surfactant, can greatly exceed the amount of the hydrophobic surfactant alone that can be solubilized using the same amount of the hydrophilic surfactant.

In addition to forming a thermodynamically stable aqueous dispersion upon mixing with an aqueous medium, the present compositions may also form optically clear, meta-stable or supersaturated dispersions with respect to the therapeutic agent and/or the triglyceride/hydrophobic surfactant in an amount in excess of the equilibrium solubility of the aqueous dispersion. Super-saturated solutions, whether characterized as homogeneous, single-phase and clear aqueous dispersions (as initially formed), or as multi-phase solutions (as would be expected if the meta-stable state breaks down), are also within the scope of the present invention. It is particularly desirable, however, that a meta-stable or supersaturated composition containing the therapeutic agent, triglyceride, and/or the hydrophobic surfactant is formed in the aqueous dispersion for at least a period of time sufficient for the absorption of the therapeutic agent in vivo. A suitable time period will be known by one of ordinary skill in the art. Generally, up to about eight hours, more typically from about one to about four hours, upon forming the dispersion is a sufficient time period for absorption of the therapeutic agent in vivo.

The unique pharmaceutical compositions and methods of the present invention present a number of significant and unexpected advantages, including:

Increased safety: The present compositions and methods allow for increased levels of triglyceride relative to hydrophilic surfactants, thereby reducing the need for excessively large amounts of hydrophilic surfactant. Further, the triglyceride-containing compositions of the present invention present small particle sizes, thus avoiding the problems of large particle size in conventional triglyceride-containing formulations and the concomitant safety concerns in parenteral administration.

Efficient transport: The particle sizes in the aqueous dispersions of the present invention are much smaller than the larger particles characteristic of vesicular, emulsion or microemulsion phases. This reduced particle size enables more efficient drug transport through the intestinal aqueous boundary layer, and through the absorptive brush border membrane. More efficient transport to absorptive sites leads to improved and more consistent absorption of therapeutic agents.

Less-dependence on lipolysis: The lack of large particle-size triglyceride components provides pharmaceutical compositions less dependent upon lipolysis, and upon the many poorly characterized factors that affect the rate and extent of lipolysis, for effective presentation of a therapeutic agent to an absorptive site. Such factors include the presence of composition components that may inhibit lipolysis; patient conditions which limit production of lipase, such as pancreatic lipase secretory diseases; and dependence of lipolysis on stomach pH, endogenous calcium concentration, and presence of co-lipase or other digestion enzymes. The reduced lipolysis dependence further provides transport that is less prone to suffer from any lag time between administration and absorption caused by the lipolysis process, enabling a more rapid onset of therapeutic action and better bioperformance characteristics. In addition, pharmaceutical compositions of the present invention can make use of hydrophilic surfactants that might otherwise be avoided or limited due to their potential lipolysis inhibiting effects.

Non-dependence on bile and meal fat contents: Due to the higher solubilization potential over bile salt micelles, the present compositions are less dependent on endogenous bile and bile related patient disease states, and meal fat contents. These advantages overcome meal-dependent absorption problems caused by poor patient compliance with meal-dosage restrictions.

Superior solubilization: The triglyceride and surfactant combinations used in compositions of the present invention enable superior loading capacity over conventional formulations. Thus, for example, more therapeutic agent can be solubilized in the triglyceride and surfactant combinations described herein than would be possible with conventional formulations containing only surfactant alone. Stated differently, the presence of the triglyceride in the present combinations improves the loading of the therapeutic agent for any given surfactant level. In addition, the particular combination of surfactants used can be optimized for a specific therapeutic agent to more closely match the polarity distribution of the therapeutic agent, resulting in still further enhanced solubilization.

Superior loading/presentation of absorption enhancers: The triglyceride and surfactant combinations in the present compositions enhance the compositions' loading capacity with respect to absorption enhancers incorporated therein, and also provide for superior presentation of the enhancers at the absorption sites, relative to conventional formulations. Consequently, the invention also includes a method for increasing the loading capacity of a pharmaceutical composition by providing: a pharmaceutical composition comprised of (a) a carrier comprising a triglyceride and a first surfactant, and (b) a therapeutically effective amount of a polysaccharide drug; and adding an absorption-enhancing amount of a second surfactant to the pharmaceutical composition, the second surfactant comprising a hydrophobic surfactant, wherein the absorption-enhancing amount is effective to increase the loading capacity of the pharmaceutical composition. The formulations formed by the method form clear aqueous dispersions. The formed dispersions preferably have, for example, an average particle size of less than about 200 nm and an absorbance of less of less than about 0.3 at 400 nm.

Preferred absorption enhancers include, without limitation, those mentioned in the overviews provided by Muranishi (1990), “Absorption Enhancers,” Critical Reviews in Therapeutic Drug Carrier Systems 7 (1):1-33; Aungst (2000), “Intestinal Permeation Enhancers,” J. Pharm. Sci. 89(4):429-442 and Curatolo et al. “Safety Assessment of Intestinal Permeability Enhancers” in “Drug Absorption Enhancement” (ed.) Boer, Harwood Academic Publishers. Such absorption enhancers include, for example, fatty acids, e.g., as described in sections 2.1, 2.2, 2.3, 2.4, 2.6, 2.7, 2.11, 2.16 and 2.19 supra, monoglycerides e.g., as described in section 2.9 supra, lecithins, e.g., as described in section 2.18 supra, and bile acids, e.g., as described in section 2.19 supra.

Because the compositions of the present invention provide a clear aqueous dispersion upon mixing with an aqueous medium, they have the advantage of sufficiently solubilizing and effectively presenting any absorption enhancer present in the compositions in an absorption-enhancing form at the absorption site of the therapeutic agent. For example, chenodeoxycholic acid (CDCA) and ursodeoxycholic acid (UDCA) are known enhancers for promoting the oral absorption of macromolecules. CDCA and UDCA, particularly UDCA, is practically insoluble in water having a pH at about 7 and below. As a result, there is a high probability that these enhancers will exist in their insoluble forms in the stomach and duodenum, thereby limiting their absorption-enhancing activity in a conventional formulation. The compositions of the present invention, however, are advantageous in that the absorption enhancer remains solubilized in the aqueous environment of the stomach and/or intestines following oral administration of the composition.

As another example, glycerol monooleate, like other hydrophobic enhancers, is practically water insoluble. In the absence of sufficient dispersion and/or solubilization, glycerol monooleate compositions form a turbid and coarse emulsion of large oil droplets that have little absorption enhancement activity. However, the combination of triglyceride and surfactants of the present invention enables the solubilization of glycerol mono-oleate in a clear aqueous dispersion, thereby facilitating the absorption-enhancing ability of glycerol monooleate.

Faster dissolution and release: Due to the robustness of compositions of the present invention to dilution, the therapeutic agents remain solubilized and thus do not suffer problems of precipitation of the therapeutic agent in the time frame relevant for absorption. In addition, the therapeutic agent is presented in small particle carriers, and is not limited in dilution rate by entrapment in emulsion carriers. These factors avoid liabilities associated with the poor partitioning of lipid solubilized drug in to the aqueous phase, such as large emulsion droplet surface area, and high interfacial transfer resistance, and enable rapid completion of the critical partitioning step.

Consistent performance: Aqueous dispersions of the present invention are thermodynamically stable for the time period relevant for absorption, and can be more predictably reproduced, thereby limiting variability in bioavailability—a particularly important advantage for therapeutic agents with a narrow therapeutic index.

Efficient release: The compositions of the present invention are designed with components that help to keep the therapeutic agent or absorption promoter, such as a permeation enhancer, an enzyme inhibitor, etc., solubilized for transport to the absorption site, but readily available for absorption, thus providing a more efficient transport and release.

Less prone to gastric emptying delays: Unlike conventional triglyceride-containing formulations, the present compositions are less prone to gastric emptying delays, resulting in faster absorption. Further, the particles in dispersions of the present invention are less prone to unwanted retention in the gastrointestinal tract.

Small size: Because of the small particle size in aqueous dispersion, the pharmaceutical compositions of the present invention allow for faster transport of the therapeutic agent through the aqueous boundary layer. Thus, the triglyceride and surfactants of the carrier are present in amounts such that upon mixing with an aqueous medium in an aqueous medium to carrier ratio of about 100:1 by weight, the carrier forms an aqueous dispersion having an average particle size less than about 200 nm.

Improved delivery of the therapeutic agent: As discussed previously, the delivery of the therapeutic agent is improved with respect to the extent, rate, and/or consistency of the absorption of the therapeutic agent. The improved delivery is a result of improved loading and solubilization of the triglyceride, the surfactant, and/or the therapeutic agent in the present compositions and in the aqueous dispersions thereof, as indicated, for example, by the clarity of the aqueous dispersion. In one approach, the delivery of the therapeutic agent is enhanced as a result of an increased amount of the therapeutic agent in a readily absorbable form. Delivery of hydrophobic therapeutic agents, may be enhanced based on this approach.

In another approach, the delivery of the therapeutic agent can also be potentially enhanced by the improved permeability of the therapeutic agent across the absorption barrier, e.g., the mucosal membranes in the nasal cavity, in the oral cavity, in the gastrointestinal tract, in the lungs and elsewhere in the body. Improved permeability is a result of improved loading in the composition, improved solubilization in the aqueous dispersion of the composition at the site of absorption, and the hydrophobic surfactants, e.g. fatty acids, bile acids, and mono-, di-fatty acid esters of polyols such as propylene glycol, glycerol, and polyethylene glycol, which often have absorption-enhancer activity. Delivery of hydrophilic therapeutic agents, such as bisphosphonates, low molecular weight heparin, oligonucleotides, and insulin may exhibit enhanced delivery based on this approach.

These and other advantages of the present invention, as well as aspects of preferred embodiments, are illustrated more fully in the Examples that follow.

### EXAMPLES

### Example 1

### Preparation of Compositions

A simple pre-concentrate is prepared as follows. Predetermined weighed amounts of the surfactants and triglyceride are stirred together to form a homogeneous mixture. For combinations that are poorly miscible, the mixture can be gently heated to aid in formation of the homogeneous mixture. If the composition is to include a therapeutic agent, the chosen therapeutic agent in a predetermined amount is added and stirred until solubilized. Optionally, solubilizers or additives are included by simple mixing.

To form an aqueous dispersion of the pre-concentrate, a predetermined amount of purified water, buffer solution, or aqueous simulated physiological solution, is added to the pre-concentrate, and the resultant mixture is stirred to form a clear, aqueous dispersion.

### Example 2

### Triglyceride Solubilization in Conventional Formulations

Conventional formulations of a triglyceride and a hydrophilic surfactant were prepared for comparison to compositions of the present invention. For each surfactant-triglyceride pair, multiple dispersions were prepared with differing amounts of the two components, to determine the maximum amount of the triglyceride that can be present while the composition still forms a clear dispersion upon a 100-fold dilution with distilled water. No therapeutic agent was included in these compositions, since it is believed that the presence of the therapeutic agent does not substantially affect the clear, aqueous nature of composition. For the same reason, these compositions were free of additional solubilizers and other additives. The optical clarity was determined by visual inspection and/or by UV absorption (at 400 nm). When UV absorption was used, compositions were considered to be clear when the absorption was less than about 0.2. Table 20 shows the maximum amount of triglyceride present in such binary mixtures forming clear aqueous dispersions. The numerical entries in the Table are in units of grams of triglyceride per 100 grams of hydrophilic surfactant.

TABLE 20
Binary Triglyceride-Surfactant Solubility

| | | | | | PEG-45 | | |
|---|---|---|---|---|---|---|---|
| | | | | | Palm | | |
| | PEG-35 | PEG-40H | | PEG-60 | Kernel | | |
| Hydrophilic | Castor Oil | Castor-Oil | PEG-6 Caprate/ | Corn Oil | Oil | Polysorbate | Polysorbate |
| Surfactant | (Incrocas | (Cremophor | Caprylate | (Crovol | (Crovol | 20 (Tween | 80 (Tween |
| Triglyceride | 35) | RH-40) | (Softigen767) | M-70) | PK-70) | 20) | 80) |
| Corn Oil (Croda, | 10 | 25 | 3 | 5 | 8 | 2 | 10 |
| Super Refined) | | | | | | | |
| Soybean Oil | 10 | 25 | 3 | 8 | 8 | 2 | 10 |
| (Croda, Super | | | | | | | |
| Refined) | | | | | | | |
| Glyceryl | 60 | 40 | 8 | 30 | 25 | 20 | 45 |
| Tricaprylate/ | | | | | | | |
| Caprate (Captex | | | | | | | |
| 300) | | | | | | | |
| Glyceryl | 70 | 40 | 5 | 55 | 30 | 20 | 55 |
| Tricaprylate/Caprate | | | | | | | |
| (Captex 355) | | | | | | | |
| Glyceryl | 70 | 60 | 5 | 55 | 25 | 10 | 50 |
| Tricaprylate/Caprate/ | | | | | | | |
| Laurate | | | | | | | |
| (Captex 350) | | | | | | | |
| Glyceryl | 30 | 40 | 3 | 25 | 15 | 2 | 25 |
| Tricaprylate/Caprate/ | | | | | | | |
| Linoleate | | | | | | | |
| (Captex 810D) | | | | | | | |

### Example 3

### Effect of Surfactant Combinations

The procedure of Example 2 was repeated for compositions containing PEG-40 hydrogenated castor oil (Cremophor RH 40) or polysorbate 80 (Tween 80) as the hydrophilic surfactant, but substituting a second hydrophilic surfactant (compositions number 6-7 and 14-16) or a hydrophobic surfactant (compositions number 4-5, 8-9, and 17-18) for part of the hydrophilic surfactant. The total amount of hydrophilic surfactant 10 was kept constant. The results are summarized in Table 21.

TABLE 21
TABLE 21A
Effects of Surfactant Combinations on the Solubilization of Triglycerides

| | Composition in w/w ratio | | | | | | | | |
|---|---|---|---|---|---|---|---|---|---|
| | 1 | 2 | 3 | 4 | 5 | 6 | 7 | 8 | 9 |
| Corn Oil | 25 | 30 | 40 | 40 | 40 | 40 | 40 | 40 | 40 |
| Cremophor | 100 | 100 | 100 | 77 | 71 | 67 | 57 | 62 | 57 |
| RH-40 | | | | | | | | | |
| Peceol | — | — | — | 23 | 29 | — | — | — | — |
| Kessco PEG | — | — | — | — | — | 33 | 43 | — | — |
| 400 MO | | | | | | | | | |
| Crovol M-40 | — | — | — | — | — | — | — | 38 | 43 |
| Appearance | Clear | Hazy | Hazy | Clear | Clear | Clear | Clear | Hazy | Hazy |
| of the | | | | | | | | | |
| Concentrate | | | | | | | | | |
| Abs @ 400 nm | 0.148 | 2.195 | 2.518 | 0.121 | 0.132 | 0.124 | 0.102 | 0.233 | 0.167 |
| of the | | | | | | | | | |
| 100x (w/v) | | | | | | | | | |
| Dilution in | | | | | | | | | |
| Deionized | | | | | | | | | |
| Water | | | | | | | | | |

TABLE 21B
Effects of Surfactant Combinations on the Solubilization of Triglycerides

| | Composition in w/w ratio | | | | | | | | |
|---|---|---|---|---|---|---|---|---|---|
| | 10 | 11 | 12 | 13 | 14 | 15 | 16 | 17 | 18 |
| Corn Oil | 10 | 15 | 20 | 30 | 15 | 20 | 30 | 20 | 25 |
| Cremophor | 100 | 100 | 100 | 100 | 67 | 67 | 67 | 67 | 67 |
| RH-40 | | | | | | | | | |
| Kessco PEG | — | — | — | — | 33 | 33 | 33 | — | — |
| 400 MO | | | | | | | | | |
| Crovol M-40 | — | — | — | — | — | — | — | 33 | 33 |
| Appearance | Clear | Clear | Hazy | Hazy | Clear | Clear | Clear | Clear | Clear |
| of the | | | | | | | | | |
| Concentrate | | | | | | | | | |
| Abs @ 400 nm | 0.002 | 1.314 | 1.613 | 1.654 | 0.041 | 0.019 | 0.194 | 0.057 | 0.158 |
| of the | | | | | | | | | |
| 100x (w/v) | | | | | | | | | |
| Dilution in | | | | | | | | | |
| Deionized | | | | | | | | | |
| Water | | | | | | | | | |

The clear or hazy appearance noted in the Table is that of the pre-concentrate, not of the aqueous dispersion. The clarity of the aqueous dispersion is shown quantitatively by UV absorption of the 1000× dilution at 400 nm.

Comparing compositions 1-3, a binary corn oil-Cremophor RH-40 mixture having 25 grams of corn oil per 100 grams of the surfactant is optically clear, having an absorption of 0.148. However, upon a slight increase of the amount of corn oil to 30 grams, the dispersion becomes cloudy, with an absorbance of 2.195, indicating the formation of a conventional emulsion. Compositions 4-5 show the surprising result that when part of the hydrophilic Cremophor RH-40 is replaced by a hydrophobic surfactant (Peceol), keeping the total surfactant concentration constant, compositions having a much higher amount of triglyceride (40 grams) still form clear aqueous dispersions, with absorbances less than 0.2 and dramatically less than the comparable binary composition number 3. A similar result is shown in compositions 8-9 for a different hydrophobic surfactant, Crovol M-40. Likewise, when part of the hydrophilic surfactant is replaced by a second hydrophilic surfactant in compositions 6-7, it is surprisingly found that the amount of triglyceride solubilized is similarly increased.

The second part of the Table, Table 21B, shows a similar surprising result for a different hydrophilic surfactant, Tween 80. Simple binary corn oil-Tween 80 mixtures form clear aqueous dispersions with 10 grams of corn oil, but are cloudy and multi-phasic with 15 grams or more of the triglyceride. As the Table shows, substitution of part of the hydrophilic surfactant with a second hydrophilic surfactant or a hydrophobic surfactant dramatically increases the amount of triglyceride that can be solubilized.

### Example 4

### Effect of Surfactant Combinations

Example 3 was repeated, using different triglyceride-surfactant combinations. In particular, medium-chain triglycerides (MCTs) were used instead of corn oil, a long-chain triglyceride (LCT). The results are shown in the three-part Table 22.

TABLE 22
TABLE 22A
Solubilization of MCTs

| | Composition in w/w ratio | | | | |
|---|---|---|---|---|---|
| | 19 | 20 | 21 | 22 | 23 |
| Pureco 76 | 33 | 50 | 80 | 50 | 80 |
| Cremophor RH-40 | 100 | 100 | 100 | 40 | 100 |
| Imwitor 988 | — | — | — | 60 | 100 |
| Ethanol | — | — | — | — | 33 |
| Appearance | Clear | Clear | Hazy | Clear | Clear |
| of the | | | | | |
| Concentrate | | | | | |
| Abs @ 400 nm | 0.201 | 0.346 | 2.522 | 0.204 | 0.098 |
| of the | | | | | |
| 100x (w/v) | | | | | |
| Dilution in | | | | | |
| Deionized | | | | | |
| Water | | | | | |

TABLE 22B
Solubilization of MCTs

| | Composition in w/w ratio | | | |
|---|---|---|---|---|
| | 24 | 25 | 26 | 27 |
| Captex 300 | 40 | 75 | 75 | 75 |
| Cremophor RH-40 | 100 | 100 | 50 | 100 |
| Imwitor 988 | — | — | 50 | 75 |
| Appearance of | Clear | Hazy | Clear | Clear |
| the Concentrate | | | | |
| Abs @ 400 nm | 0.180 | 0.557 | 2.208 | 0.078 |
| of the 100x (w/v) | | | | |
| Dilution in | | | | |
| Deionized Water | | | | |

TABLE 22C
Solubilization of MCTs

| | Composition in w/w ratio | | | | | |
|---|---|---|---|---|---|---|
| | 28 | 29 | 30 | 31 | 32 | 33 |
| Captex 300 | 20 | 25 | 33 | 30 | 40 | 40 |
| Tween 20 | 100 | 100 | 100 | 70 | 70 | 66 |
| Brij 30 | — | — | — | 30 | 30 | 34 |
| Appearance | Clear | Hazy | Hazy | Clear | Clear | Clear |
| of the | | | | | | |
| Concentrate | | | | | | |
| Abs @ 400 nm | 0.078 | 1.192 | 2.536 | 0.017 | 0.234 | 0.103 |
| of the | | | | | | |
| 100x (w/v) | | | | | | |
| Dilution in | | | | | | |
| Deionized | | | | | | |
| Water | | | | | | |

Table 22 shows that the increased solubilization of the triglyceride is observed for MCTs as well as for LCTs, with a variety of surfactants. Table 22 additionally shows that the same effect is observed in the presence of increased amounts of surfactants (compositions 23 and 27) and solubilizers (composition 23).

### Example 5

### Characterization of Compositions

Various compositions were prepared and characterized by visual observation as well as by UV absorbance at 400 nm. Each composition was diluted 100-fold with distilled water. The results are shown in Table 23.

TABLE 23
Visual and Spectroscopic Characterization

| | | | Visual | Absorbance at |
|---|---|---|---|---|
| No. | Composition | | Observation | 400 nm |
| 24 | Soybean Oil | 80 mg | Very clear | 0.014 |
| | Tween 20 | 200 mg | solution | |
| | Tween 80 | 800 mg | | |
| 25 | Captex 810D | 250 mg | Very clear | 0.030 |
| | Incrocas 35 | 500 mg | solution | |
| | Tween 80 | 500 mg | | |
| 26 | Captex 810D | 200 mg | Clear solution | 0.157 |
| | Incrocas 35 | 667 mg | | |
| | Myvacet 9-45 | 333 mg | | |
| 27 | Corn Oil | 250 mg | Clear solution | 0.085 |
| | Cremophor RH- | 750 mg | | |
| | 40 | | | |
| | Peceol | 150 mg | | |
| | Propylene Glycol | 100 mg | | |
| 28 | Captex 355 | 200 mg | Clear solution | 0.212 |
| | Labrafil | 300 mg | | |
| | M2125CS | | | |
| | Cremophor RH- | 500 mg | | |
| | 40 | | | |
| | Ethanol | 100 mg | | |
| 29 | Captex 355 | 150 mg | Clear solution | 0.141 |
| | Cremophor RH- | 600 mg | | |
| | 40 | | | |
| | Labrafil | 250 mg | | |
| | M2125CS | | | |
| | Ethanol | 100 mg | | |
| 30 | Captex 355 | 300 mg | Clear solution, | 0.241 |
| | Incrocas 35 | 500 mg | Slightly hazy | |
| | Labrafil | 200 mg | | |
| | M2125CS | | | |
| | Ethanol | 100 mg | | |
| 31 | Captex 355 | 250 mg | Clear solution | 0.076 |
| | Incrocas 35 | 600 mg | | |
| | Labrafil | 150 mg | | |
| | M2125CS | | | |
| | Ethanol | 100 mg | | |
| 32 | Pureco 76 | 160 mg | Clear solution | 0.168 |
| | Cremophor RH- | 480 mg | | |
| | 40 | | | |
| | Labrafil | 160 mg | | |
| | M2125CS | | | |
| | Ethanol | 150 mg | | |

### Example 6

### Comparative Example

Prior art formulations were prepared for comparison with the compositions of the present invention. As in Example 5, the compositions were diluted 100-fold with distilled water, and characterized by visual observation and by UV absorbance The results are shown in Table 24.

TABLE 24
Compositions Not Forming Clear Aqueous Dispersions

| | | | Visual | Absorbance at |
|---|---|---|---|---|
| No. | Composition | | Observation | 400 nm |
| 33 | Corn Oil | 400 mg | Milky | 1.989 |
| | Cremophor RH- | 710 mg | suspension | |
| | 40 | | | |
| | Crovol M-40 | 290 mg | | |
| 34 | Captex 300 | 300 mg | Milky | 1.594 |
| | Tween 20 | 650 mg | suspension | |
| | Imwitor 988 | 350 mg | | |
| 35 | Corn Oil | 400 mg | Milky | 2.716 |
| | Cremophor RH- | 620 mg | suspension | |
| | 40 | | | |
| | Labrafil | 380 mg | | |
| | M2125CS | | | |
| 36 | Soybean Oil | 185 mg | Milky | 2.595 |
| | Captex GTO | 275 mg | suspension | |
| | Tween 80 | 275 mg | | |
| | Triacetin | 185 mg | | |
| 37 | Pureco 76 | 315 mg | Milky | 2.912 |
| | Cremophor RH- | 225 mg | suspension | |
| | 40 | | | |
| | Span 20 | 360 mg | | |
| 38 | Soybean Oil | 340 mg | Milky | 2.566 |
| | Captex GTO | 280 mg | suspension | |
| | Tween 80 | 280 mg | | |
| 39 | Pureco 76 | 330 mg | Milky | 2.233 |
| | Labrasol | 120 mg | suspension | |
| 40 | Corn Oil | 400 mg | Milky | 2.249 |
| | Cremophor RH- | 570 mg | suspension | |
| | 40 | | | |
| | Lauroglycol FCC | 430 mg | | |
| 41 | Soybean Oil | 160 mg | Milky | 2.867 |
| | Tween 80 | 200 mg | suspension | |
| | Imwitor 988 | 450 mg | | |
| | Ethanol | 150 mg | | |
| 42 | Corn Oil | 200 mg | Milky | 1.547 |
| | Tween 80 | 570 mg | suspension | |
| | Kessco PEG 400 | 430 mg | | |
| | MO | | | |

### Example 7

### Comparative Example

TABLE 25
Formulations

| No. | Composition | (g) |
|---|---|---|
| 43 | Cremophor RH-40 | 0.75 |
| | Peceol | 0.25 |
| | Corn Oil NF | 0.40 |
| | Fenofibrate | 0.10 |
| 44 | Cremophor RH-40 | 0.57 |
| | Crovol M-40 | 0.43 |
| | Corn Oil NF | 0.40 |
| | Rofecoxib | 0.15 |
| 45 | Cremophor RH-40 | 0.57 |
| | Kessco PEG 400 MO | 0.43 |
| | Soybean Oil NF | 0.40 |
| | Nabumetone | 0.30 |
| 46 | Tween 80 | 0.70 |
| | Tween 85 | 0.35 |
| | Miglyol 812 | 0.30 |
| | Paclitaxel | 0.10 |
| | PEG 400 | 0.25 |
| 47 | Cremophor RH-40 | 0.50 |
| | Imwitor 988 | 0.50 |
| | Captex 300 | 0.75 |
| | Cyclosporin A | 0.20 |
| | Propylene Glycol | 0.15 |
| 48 | Tween 20 | 0.66 |
| | Brij 30 | 0.34 |
| | Captex 355 | 0.40 |
| | Retinoic Acid | 0.02 |
| 49 | Tween 80 | 0.67 |
| | Kessco PEG 400 MO | 0.33 |
| | Corn Oil | 0.30 |
| | Terbinafine | 0.25 |
| 50 | Crovol M-40 | 0.67 |
| | Crovol M-40 | 0.33 |
| | Captex 350 | 0.75 |
| | Progesterone | 0.10 |
| | Ethanol | 0.15 |
| 51 | Labrasol | 0.30 |
| | Gelucire 44/14 | 0.70 |
| | Dronabinol | 0.02 |
| | Ethanol | 0.10 |
| 52 | Incrocas 35 | 0.80 |
| | Arlacel 186 | 0.20 |
| | Miglyol 818 | 0.45 |
| | Alendronate sodium | 0.04 |
| | Water | 0.10 |
| 53 | Cremophor RH-40 | 0.62 |
| | Capmul MCM | 0.38 |
| | Miglyol 810 | 0.25 |
| | Heparin sodium | 0.03 |
| | Water | 0.10 |
| | PEG 400 | 0.05 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising:
a) a carrier comprising:
i) a triglyceride;
ii) at least two surfactants, at least one of the surfactants being a hydrophilic surfactant; and
b) a therapeutically effective amount of a sex hormone;
wherein the triglyceride and surfactants are present in amounts such that upon mixing with an aqueous solution in an aqueous solution to a composition ratio of about 100:1 by weight, the composition forms a dispersion having an average particle size of less than 200 nm.

2. The pharmaceutical composition of claim 1, wherein the sex hormone is progesterone, testosterone or an ester thereof.

3. The pharmaceutical composition of claim 1, wherein the carrier enhances the bioabsorption of the sex hormone.

4. The pharmaceutical composition of claim 1, wherein the carrier increases the rate and extent of absorption of the sex hormone.

5. The pharmaceutical composition of claim 1, wherein the carrier provides for a more rapid onset of therapeutic action, better bioperformance characteristics, or a combination thereof of the sex hormone.

6. The pharmaceutical composition of claim 1, wherein the triglyceride is selected from the group consisting of vegetable oils, fish oils, animal fats, hydrogenated vegetable oils, partially hydrogenated vegetable oils, synthetic triglycerides, fractionated triglycerides, and mixtures thereof.

7. The pharmaceutical composition of claim 1, wherein the hydrophilic surfactant comprises at least one non-ionic hydrophilic surfactant having an HLB value greater than or equal to about 10.

8. The pharmaceutical composition of claim 7, wherein the non-ionic surfactant is selected from the group consisting of alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; sugar esters, sugar ethers; sucroglycerides; and mixtures thereof.

9. The pharmaceutical composition of claim 7, wherein the non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

10. The pharmaceutical composition of claim 9, wherein the polyol is selected from the group consisting of glycerol, ethylene glycol, polyethylene glycol, sorbitol, propylene glycol, pentaerythritol, a saccharide, and mixtures thereof.

11. The pharmaceutical composition of claim 1, the hydrophilic surfactant is selected from the group consisting of PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, PEG-24 cholesterol, a poloxamer, and mixtures thereof.

12. The pharmaceutical composition of claim 1, wherein the carrier comprises at least one hydrophilic surfactant and at least one hydrophobic surfactant.

13. The pharmaceutical composition of claim 12, wherein the hydrophobic surfactant is selected from the group consisting of oleic acid; lauric acid; glyceryl monocaprate; glyceryl monocaprylate; glyceryl monolaurate; glyceryl monooleate; glyceryl dicaprate; glyceryl dicaprylate; glyceryl dilaurate; glyceryl dioleate; acetylated monoglycerides; propylene glycol oleate; propylene glycol laurate; polyglyceryl-3 oleate; polyglyceryl-6 dioleate; PEG-6 corn oil; PEG-20 corn oil; PEG-20 almond oil; sorbitan monooleate; sorbitan monolaurate; POE-4 lauryl ether; POE-3 oleyl ether; ethyl oleate; poloxamers; cholic acid; ursodeoxycholic acid; glycocholic acid; taurocholic acid; lithocholic acid; deoxycholic acid; chenodeoxycholic acid; and mixtures thereof.

14. The pharmaceutical composition of claim 1, wherein the triglyceride and surfactants are present in amounts such that upon mixing with an aqueous solution in an aqueous solution to composition ratio of about 10:1 by weight, the composition forms a clear aqueous dispersion.

15. The pharmaceutical composition of claim 1, which further comprises at least one additive selected from the group consisting of an antioxidant, a bufferant, an antifoaming agent, a detackifier, a preservative, a chelating agent, a viscomodulator, a tonicifier, a flavorant, a colorant, an odorant, an opacifier, a suspending agent, a binder, a filler, a plasticizer, and a lubricant.

16. The pharmaceutical composition of claim 1, wherein the composition has a triglyceride to surfactant ratio of 2:100 to 75:100.

17. The pharmaceutical composition of claim 1, wherein the composition has a triglyceride to surfactant ratio of 15:100 to 75:100.

18. A dosage form comprising the pharmaceutical composition of claim 1, wherein the dosage form is selected from the group consisting of a pill, capsule, caplet, tablet, granule, bead and powder.

19. A dosage form comprising the pharmaceutical composition of claim 1, wherein the dosage form is selected from the group consisting of a solution, suspension, emulsion, cream, ointment, lotion, suppository, spray, aerosol, paste, gel, drops, douche, ovule, wafer, troche, cachet, syrup and elixir.

20. The pharmaceutical composition of claim 2, wherein the testosterone ester is testosterone enanthate, testosterone proprionate, testosterone undecanoate, testosterone palmitate, or a combination thereof.

21. The pharmaceutical composition of claim 2, wherein the testosterone ester is testosterone enanthate.

22. The pharmaceutical composition of claim 2, wherein the testosterone ester is testosterone proprionate.

23. The pharmaceutical composition of claim 2, wherein the testosterone ester is testosterone undecanoate.

24. The pharmaceutical composition of claim 2, wherein the testosterone ester is testosterone palmitate.

25. A pharmaceutical composition comprising:
a) a triglyceride;
b) a carrier comprising at least two surfactants, at least one of the surfactants being hydrophilic; and
c) a therapeutically effective amount of a sex hormone.

26. The pharmaceutical composition of claim 25, wherein the carrier enhances the bioabsorption of the sex hormone.

27. The pharmaceutical composition of claim 25, wherein the carrier increases the rate and extent of absorption of the sex hormone.

28. The pharmaceutical composition of claim 25, wherein the carrier provides for a more rapid onset of therapeutic action, better bioperformance characteristics, or a combination thereof of the sex hormone.

29. The pharmaceutical composition of claim 25, wherein the triglyceride is selected from the group consisting of vegetable oils, fish oils, animal fats, hydrogenated vegetable oils, partially hydrogenated vegetable oils, synthetic triglycerides, fractionated triglycerides, and mixtures thereof.

30. The pharmaceutical composition of claim 25, wherein the hydrophilic surfactant comprises at least one non-ionic hydrophilic surfactant having an HLB value greater than or equal to about 10.

31. The pharmaceutical composition of claim 30, wherein the non-ionic surfactant is selected from the group consisting of alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; sugar esters, sugar ethers; sucroglycerides; and mixtures thereof.

32. The pharmaceutical composition of claim 30, wherein the non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

33. The pharmaceutical composition of claim 32, wherein the polyol is selected from the group consisting of glycerol, ethylene glycol, polyethylene glycol, sorbitol, propylene glycol, pentaerythritol, a saccharide, and mixtures thereof.

34. The pharmaceutical composition of claim 25, the hydrophilic surfactant is selected from the group consisting of PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, PEG-24 cholesterol, a poloxamer, and mixtures thereof.

35. The pharmaceutical composition of claim 25, wherein the carrier comprises at least one hydrophilic surfactant and at least one hydrophobic surfactant.

36. The pharmaceutical composition of claim 35, wherein the hydrophobic surfactant is selected from the group consisting of oleic acid; lauric acid; glyceryl monocaprate; glyceryl monocaprylate; glyceryl monolaurate; glyceryl monooleate; glyceryl dicaprate; glyceryl dicaprylate; glyceryl dilaurate; glyceryl dioleate; acetylated monoglycerides; propylene glycol oleate; propylene glycol laurate; polyglyceryl-3 oleate; polyglyceryl-6 dioleate; PEG-6 corn oil; PEG-20 corn oil; PEG-20 almond oil; sorbitan monooleate; sorbitan monolaurate; POE-4 lauryl ether; POE-3 oleyl ether; ethyl oleate; poloxamers; cholic acid; ursodeoxycholic acid; glycocholic acid; taurocholic acid; lithocholic acid; deoxycholic acid; chenodeoxycholic acid; and mixtures thereof.

37. The pharmaceutical composition of claim 25, which further comprises at least one additive selected from the group consisting of an antioxidant, a bufferant, an antifoaming agent, a detackifier, a preservative, a chelating agent, a viscomodulator, a tonicifier, a flavorant, a colorant, an odorant, an opacifier, a suspending agent, a binder, a filler, a plasticizer, and a lubricant.

38. The pharmaceutical composition of claim 25, wherein the composition has a triglyceride to surfactant ratio of 2:100 to 75:100.

39. The pharmaceutical composition of claim 25, wherein the composition has a triglyceride to surfactant ratio of 15:100 to 75:100.

40. A dosage form comprising the pharmaceutical composition of claim 25, wherein the dosage form is selected from the group consisting of a pill, capsule, caplet, tablet, granule, bead and powder.

41. A dosage form comprising the pharmaceutical composition of claim 25, wherein the dosage form is selected from the group consisting of a solution, suspension, emulsion, cream, ointment, lotion, suppository, spray, aerosol, paste, gel, drops, douche, ovule, wafer, troche, cachet, syrup and elixir.

42. The pharmaceutical composition of claim 25, wherein the sex hormone is progesterone, testosterone, or an ester thereof.

43. The pharmaceutical composition of claim 25, wherein the sex hormone is testosterone enanthate, testosterone proprionate, testosterone undecanoate, testosterone palmitate, or a combination thereof.

44. The pharmaceutical composition of claim 42, wherein the testosterone ester is testosterone enanthate.

45. The pharmaceutical composition of claim 42, wherein the testosterone ester is testosterone proprionate.

46. The pharmaceutical composition of claim 42, wherein the testosterone ester is testosterone undecanoate.

47. The pharmaceutical composition of claim 42, wherein the testosterone ester is testosterone palmitate.

48. The pharmaceutical composition of claim 25, wherein upon dilution of the composition, the active agent increases the extent of dispersion of the vitamin E substance by at least 20% relative to the dispersion of the composition without the active agent.

49. A pharmaceutical composition comprising:
a) a triglyceride;
b) a hydrophilic alcohol transesterified surfactant; and
c) a therapeutically effective amount of testosterone, or an ester thereof.

50. The pharmaceutical composition of claim 49, wherein the triglyceride is selected from the group consisting of vegetable oils, fish oils, animal fats, hydrogenated vegetable oils, partially hydrogenated vegetable oils, synthetic triglycerides, fractionated triglycerides, and mixtures thereof.

51. The pharmaceutical composition of claim 49, wherein the hydrophilic alcohol transesterified surfactant is selected from the group consisting of PEG-35 castor oil, PEG-40 castor oil, PEG-40 hydrogenated castor oil, or a combination thereof.

52. The pharmaceutical composition of claim 49, wherein the composition has a triglyceride to surfactant ratio of 2:100 to 75:100.

53. The pharmaceutical composition of claim 49, wherein the testosterone ester is testosterone undecanoate.

54. A dosage form comprising the pharmaceutical composition of claim 49, wherein the dosage form is selected from the group consisting of a pill, capsule, caplet, tablet, granule, bead and powder.

55. A pharmaceutical composition comprising:
a) a triglyceride;
b) PEG-35 castor oil, PEG-40 castor oil, PEG-40 hydrogenated castor oil, or a combination thereof; and
c) a therapeutically effective amount of testosterone, or an ester thereof.

56. The pharmaceutical composition of claim 55, wherein the triglyceride is selected from the group consisting of vegetable oils, fish oils, animal fats, hydrogenated vegetable oils, partially hydrogenated vegetable oils, synthetic triglycerides, fractionated triglycerides, and mixtures thereof.

57. The pharmaceutical composition of claim 55, wherein the composition has a triglyceride to oil ratio of 2:100 to 75:100.

58. The pharmaceutical composition of claim 55, wherein the composition has a triglyceride to surfactant ratio of 15:100 to 75:100.

59. The pharmaceutical composition of claim 55, wherein the testosterone ester is testosterone undecanoate.

60. A dosage form comprising the pharmaceutical composition of claim 55, wherein the dosage form is selected from the group consisting of a pill, capsule, caplet, tablet, granule, bead and powder.

61. A method of providing hormone therapy in a patient comprising orally administering to a patient in need thereof a pharmaceutical composition comprising:
a) a therapeutically effective amount of sex hormone;
b) a triglyceride; and
c) a carrier comprising at least two surfactants, at least one of the surfactants being hydrophilic.

62. The method of claim 61, wherein the triglyceride and surfactants are present in amounts such that upon mixing with an aqueous solution in an aqueous solution to a composition to a composition ratio of about 100:1 by weight, the composition forms a clear aqueous dispersion having an average particle size of less than 200 nm.
